(19) United States
(12) Patent Application Publication
(10) Pub. No.: US 2018/0185327 A1
(43) Pub. Date: Jul. 5, 2018

(54) FORMULATION COMPRISING PARTICLES AND A LIPASE INHIBITOR

(71) Applicant: PERORA GMBH, Heidelberg (DE)

(72) Inventor: Dirk VETTER, Heidelberg (DE)

(21) Appl. No.: 15/742,379

(22) PCT Filed: Jul. 7, 2016
(86) PCT No.: PCT/EP2016/066216

§ 371 Date: Jan. 5, 2018

(30) Foreign Application Priority Data
Jul. 7, 2015 (EP) 15175571.7
Jul. 8, 2015 (EP) 15175819.0
Aug. 11, 2015 (EP) 15180659.3
Dec. 23, 2015 (EP) 15202552.4

(51) Int. Cl. A61K 31/365; A61K 9/14; A61K 9/50; A61K 9/48; A61K 47/12; A61K 47/32; A61K 47/36; A61K 47/14; A61K 9/00; A61P 3/04

(52) U.S. Cl. CPC A61K 31/365; A61K 9/145; A61K 9/146; A61K 9/5015; A61K 9/4866; A61K 47/12; A61P 3/04; A61K 47/36; A61K 47/14; A61K 9/0053; A61K 9/5026; A61K 9/5036; A61K 9/5078; A61K 47/32

(57) ABSTRACT

The invention provides a pharmaceutical combination product for oral administration comprising a lipase inhibitor and a plurality of ingestible particles, said particles comprising a water-swellable or water-soluble polymeric material and a lipid material. The lipase inhibitor may be provided in the ingestible particles or separate from these. The polymeric material may be embedded in the lipid material. The invention further provides methods for preparing the pharmaceutical combination product and uses thereof.

### FIELD

The present invention relates to oral compositions comprising a lipase inhibitor.

### BACKGROUND

Oral lipase inhibitors are well-established and safe medications for the treatment of overweight and obesity. The most prominent oral lipase inhibitor is orlistat (tetrahydrolipstatin). Its high-dose version is marketed as a prescription drug under the trade name Xenical® by Roche, and a low-dose version is sold over-the-counter as Alli® by GSK and has become generic. Its primary function is preventing the absorption of fats from the human diet by acting as a lipase inhibitor, thereby reducing caloric intake. Orlistat is the saturated derivative of lipstatin, a potent natural inhibitor of pancreatic lipases isolated from the bacterium Streptomyces toxytricini.

Orlistat works by inhibiting gastric and pancreatic lipases.

When lipase activity is blocked, triglycerides from the diet are not hydrolysed into absorbable free fatty acids, and are excreted undigested instead. Only trace amounts of orlistat are absorbed systemically; the primary effect is local lipase inhibition within the GI tract after an oral dose. The primary route of elimination is through the faeces.

At the standard prescription dose of 120 mg three times daily before meals, orlistat prevents approximately 30% of dietary fat from being absorbed, and about 25% at the standard over-the-counter dose of 60 mg.

As a direct consequence of the drug's efficacy in inhibiting intestinal lipase and the uptake of triglycerides, there are significant and often problematic gastro-intestinal treatment effects of the drug such as steatorrhea (oily, loose stools with excessive flatus due to unabsorbed fats reaching the large intestine), faecal incontinence and frequent or urgent bowel movements. Users should be cautious of the possible side effects until they “have a sense of any treatment effects”. To minimize these effects, foods with high fat content should be avoided; the manufacturer advises consumers to follow a low-fat, reduced-calorie diet. Oily stools and flatulence can be controlled by reducing the dietary fat content to somewhere in the region of 15 grams per meal. The manual for Alli® makes it clear that orlistat treatment involves aversion therapy, encouraging the user to associate eating fat with unpleasant treatment effects.

Another consequence of the drug's mode of action is increased hunger. It was established that orlistat has acute effects on GI function, which favour an increase, rather than a decrease, in energy intake. The presence of nutrients, especially fat, in the small intestine stimulates the release of gut hormones, including cholecystokinin (CCK), glucagon-like peptide-1 (GLP-1) and peptide YY (PYY), and suppression of ghrelin. These mediate, at least in part, the effects of fat on the reduction of hunger and subsequent energy intake, and the modulation of GI motility, leading to slowed gastric emptying, and improved glycaemic control. The effects of fat on appetite and GI function are mediated by their digestive products, free fatty acids. In the absence of free fatty acids, the satiating effect of a meal is compromised, and users tend to increase their food intake, which is counterproductive for an anti-obesity medication.

US 2008/0075688 (to Procter & Gamble) describes polymeric foams intended to sequester oil in the gastro-intestinal tract and thereby ameliorating side effects associated with oral lipase inhibitors.

WO 01/05408 (to Geltex) claims fat-binding copolymers in combination with an oral lipase inhibitor with the goal of lessening the side effect of steatorrhea.

WO 2011/096950 (to Chelatexx) outlines the combined use of simethicone and activated charcoal to cause undigested fats to remain in an emulsified state in large intestine.

EP 1572240 (to Procter & Gamble) focuses on the addition of calcium stearate to an oral lipase inhibitor for increasing the viscosity of undigested lipids in the gastro-intestinal tract.

U.S. Pat. No. 8,246,985 (to Amorepacific) mentions the use of lipophilic compounds such as hydrogenated castor oil to minimize oral lipase inhibitor side effects such as oily spotting.

It is an object of the present invention to provide oral compositions that are effective in minimizing the key side effects of lipase inhibitor orlistat: steatorrhea causing oily leakage and lack of satiety leading to increased food intake. Furthermore it is an object of the present invention to provide formulations, dosage forms and presentations of the above mentioned compositions. A yet further object is to provide a treatment for obesity which encourages adherence to the therapy and motivates the patient to comply with a prescribed administration regimen.

### SUMMARY OF THE INVENTION

In a first aspect, the invention provides a pharmaceutical combination product for oral administration comprising (i) a lipase inhibitor; and (ii) a plurality of ingestible particles having a sieve diameter in the range from 0.01 mm to 10 mm, or from 0.05 mm to 3 mm, the particles comprising (a) a water-swellable or water soluble polymeric material and (b) a first lipid material. The first lipid material comprises a medium or long chain fatty acid compound. The ingestible particles are further characterised in that the water-swellable or water-soluble polymeric material is embedded within, and/or coated with, the lipid material.

The lipase inhibitor, for example orlistat, may be contained in the ingestible particles (i.e. incorporated within the particles as material (c)) and/or it may be provided separately from them. When provided separately from the ingestible particles, the lipase inhibitor may be provided “extragranular” to said particles but in the same pharmaceutical composition; for instance in form of mixtures of the ingestible particles and the lipase inhibitor which may be compressed to tablets or filled into capsules, sachets, stick packs, bottles, or containers. Alternatively, the lipase inhibitor may also be provided in a separate pharmaceutical composition, said separate pharmaceutical composition being provided together with the plurality of ingestible particles in the form of a kit.

The first lipid material of the ingestible particles in/by which the water-swellable or water-soluble polymeric material is embedded or coated may represent the active core of the ingestible particles. The particles may further be coated with a coating layer that comprises a second lipid material and/or a hydrophilic material. Optionally, the coating layer is substantially free of the water-swellable or water-soluble polymeric material. Optionally, the active core and/or the coating may comprise a lipase inhibitor such as orlistat.

Alternatively, the ingestible particle may comprise an inert core, e.g. composed of an inert material, and the first lipid material in/by which the water-swellable or water-soluble polymeric material, and optionally a lipase inhibitor such as orlistat, is embedded or coated may be designed as a coating covering the inert core. Moreover, the particle may further comprise a second coating layer covering the first coating. The second coating comprises a second lipid material and/or a hydrophilic material, and optionally a lipase inhibitor such as orlistat. Optionally, the second coating layer is substantially free of the water-swellable or water-soluble polymeric material.

Optionally, the ingestible particles may further comprise (d) an amino acid, a vitamin, a micro-nutrient, or any combinations thereof. In this case, the water-swellable or water-soluble polymeric material (a) and/or the amino acid (d) are embedded within, or coated with, the lipid material (b). Optionally, also the vitamin(s) and/or the micronutrient(s), if present, may be embedded within, or coated with, the lipid material (b). Further optionally, the coating layers comprising the second lipid material and/or the hydrophilic material may be substantially free of the water-swellable or water-soluble polymeric material and/or the optional amino acid, vitamin and/or micro-nutrient.

The first lipid material comprises at least one medium or long chain fatty acid compound with a melting range below 37° C. and/or at least one medium or long chain fatty acid compound with a melting range above 37° C., either per se or in the hydrated state, or a mixture thereof. In one of the preferred embodiments, the melting range refers to the fatty acid glyceride component as such, i.e. not in its hydrated state. Preferably, the first lipid material comprises at least one medium or long chain fatty acid compound with a melting range above 37° C. This may prevent, or at least limit, faecal liquefaction under orlistat treatment, when lipase activity is blocked, since the triglycerides will not be hydrolysed into absorbable free fatty acids yet remain solid until being excreted. Optionally, the content of di- and triglycerides within the first lipid material may be limited; e.g. to 80% or less, or even 50% or less. This provision may help to further prevent, or at least limit, faecal liquefaction under orlistat treatment; in particular for di- and triglycerides exhibiting low melting ranges (below 37° C.), since these would remain as a molten, non-resorbable liquid inside the gut lumen until being excreted.

In a further aspect, the invention provides ingestible particles having a sieve diameter in the range from 0.01 mm to 10 mm, or from 0.05 mm to 3 mm, said particle comprising (a) a water-swellable or water-soluble polymeric material, (b) a first lipid material; and (c) a lipase inhibitor such as orlistat, and optionally (d) an amino acid, a vitamin, a micro-nutrient, or any combinations thereof, wherein the first lipid material comprises a medium or long chain fatty acid compound, and the water-swellable or water-soluble polymeric material is embedded within, and/or coated with, the lipid material.

Further optionally, the pharmaceutical combination product may comprise one or more additional constituents selected e.g. from components A to E. Component A comprises a native or modified protein; component B comprises a native or modified dietary fibre; component C comprises a vitamin, a micro-nutrient such as a micro-mineral, an organic acids, choline, cholesterol, and/or a further dietary element (also called mineral nutrients); component D comprises at least one amino acid; and component E comprises one or more substance(s) for improved flavour. Components A to E may optionally be provided in the form of a powder, a powder blend and/or a granulate.

The at least one component selected from components A to E may either be combined with the ingestible particles in the same primary packaging or dosage form as a ‘ready-to-use’ composition, or provided separately from said particles—e.g. in the form of a kit—such that the consumer, or user, may add it to the solid phase prior to ingestion.

In a further aspect, the invention provides a single dose package or container which comprises the combination product, preferably at an amount of at least about 5 g. The amount of the ingestible particles in the combination product is at least about 2 g, preferably at least about 3 g, and contains at least 1 g of the first lipid material, preferably at least 2 g of the first lipid material. The single dose package or container may for example be a vial, bottle, stick pack or sachet.

In a yet further aspect, the invention provides the use of the inventive pharmaceutical combination product or of the ingestible particles comprising the lipase inhibitor for the prevention and/or treatment of obesity, or a disease or condition which is associated with obesity and/or the use of lipase inhibitors. Moreover, the use in appetite suppression and induction of satiety is provided. The use may be associated with a dietary schedule according to which a single dose of the pharmaceutical combination product is administered to a human subject at least once a day over a period of at least one week, and wherein optionally the human subject may be instructed to substitute a meal, partially or entirely, with said administration. For instance, the pharmaceutical combination products or the ingestible particles comprising the lipase inhibitor may be used to treat or prevent lipase inhibitor induced gastro-intestinal problems.

### DETAILED DESCRIPTION OF THE INVENTION

In a first aspect, the invention provides a pharmaceutical combination product for oral administration comprising (i) a lipase inhibitor; and (ii) a plurality of ingestible particles having a sieve diameter in the range from 0.01 mm to 10 mm, or from 0.05 mm to 3 mm, the particles comprising (a) a water-swellable or water-soluble polymeric material and (b) a first lipid material. The first lipid material comprises a medium or long chain fatty acid compound. The ingestible particles (or shorter ‘particles’) are further characterised in that the water-swellable or water-soluble polymeric material is embedded within, and/or coated with, the lipid material.

In one specific embodiment of the invention, the lipase inhibitor is orlistat.

The lipase inhibitor may be contained in the ingestible particles (i.e. incorporated within the particles as a material (c)) and/or it may be provided separately from them. In this regard, the lipase inhibitor may be considered optional in the ingestible particles, as long as the final pharmaceutical combination product comprises the lipase inhibitor.

When provided separately from the ingestible particles, the lipase inhibitor may be provided “extragranular” to said particles but in the same pharmaceutical composition; for instance in form of mixtures of the ingestible particles and the lipase inhibitor which may be compressed to tablets or filled into capsules, sachets, stick packs, bottles or containers. It is to be understood that the term “extragranular” is used in the widest sense and is not intended to imply, that all ingestible particles (with or without lipase inhibitor) are necessarily prepared by a granulation step.

Alternatively, the lipase inhibitor may also be provided in a separate pharmaceutical composition, said separate pharmaceutical composition being provided together with the plurality of ingestible particles in the form of a kit.

Optionally, the ingestible particles may further comprise (d) an amino acid, a vitamin, a micro-nutrient, or any combinations thereof (i.e. incorporated within the particles).

For the avoidance of doubt, it should be understood that—unlike the lipase inhibitor—the presence of the amino acid, the vitamin, and/or the micro-nutrient in the ingestible particles (and/or mixtures for the preparation of said particles) is optional in all embodiments, unless where explicitly stated otherwise. This means that, as used herein, any listings including any of these optional components simply refer to the specific embodiments in which one or more of them are present, while not excluding those embodiments without these optional components. Where no amino acid, vitamin and/or micro-nutrient is incorporated within the ingestible particles, this does not necessarily require said optional components to be provided elsewhere in the pharmaceutical combination product.

As will be discussed in more detail further below, the pharmaceutical combination product may optionally comprise one more additional constituents selected e.g. from components A to E, with component A comprising a native or modified protein; component B a native or modified dietary fibre; component C a vitamin, a micro-nutrient such as one or more micro-minerals, organic acids, choline, cholesterol, and/or a further dietary element (also called mineral nutrients); component D at least one amino acid; and component E one or more substance(s) for improved flavour. These components A to E may optionally be provided in the form of a powder, a powder blend and/or a granulate.

The at least one component selected from components A to E may either be combined with the ingestible particles in the same primary packaging or dosage form as a ‘ready-to-use’ composition, or provided separately from said particles—e.g. in the form of a kit—such that the consumer, or user, may add it to the solid phase prior to ingestion.

The term ‘kit’ as used herein means that the components comprised in said kit are provided physically separable and distinguishable from one another as different components but are sold together for the purpose of being administered, or used, together, though not necessarily simultaneously. The kit may for instance be supplied in the form of:

a) separate compartments of one primary package (such as a sachet divided into two or more ‘sub-pouches’ by a laminating seam, or a glass vial filled with one kit component and the other kit component being held in the screw-top lid of said glass vial);

b) separate primary packages packaged together within one secondary package (such as separate sets of sachets for two or more kit components, the two or more sachet-sets being sold in one and the same folded box);

c) separate primary packages packaged in two or more separate secondary packages which are in turn held together by paper or plastic wrappers, ribbons, sleeves or the like (such as separate sets of sachets for two or more kit components, the two or more sachet-sets being sold in two or more card-board boxes, the latter being wrapped with a shrink foil wrapper); or

d) combinations thereof (such as a first kit-component being provided in multiple-dose card-board drum, optionally with a dosing spoon, the card-board drum being sold in a folded box together with a multitude of foil-wrapped single-serving sized portions of a second kit-component).

Optionally, the kits of the invention may be further comprise written instructions on how to best, or preferably, combine and use the two or more kit components.

It should also be understood that, as used herein, the terms ‘a’ or ‘an’ or ‘the’ or features described in their singular form do not exclude a plurality of the respective features. Unless explicitly stated or described otherwise, expressions such as “an amino acid”, “a water-swellable or water-soluble polymeric material”, “the first lipid material” or the like are chosen solely for reasons of simplicity and are meant to encompass one or more material(s), amino acid(s), etc.; e.g. in the form of blends, or mixtures, of two or more of the respective components.

All percentages, parts and ratios as used herein, are by weight of the total formulation, unless otherwise specified; i.e. “%” should be read as “wt.-%” unless otherwise specified or unless it is clear from the context that another type of percentage is meant.

The inventors have found that the ingestible particles as defined herein, and in particular oral combination products comprising or prepared from a plurality of the particles and a lipase inhibitor, such as orlistat, are capable of effectively inducing satiety, of suppressing the appetite, and thereby may be used to prevent or treat obesity or a disease or condition associated with obesity. Without wishing to be bound by theory, it is currently believed that upon oral administration, the fatty acid or fatty acid ester comprised in the particles as well as the lipase inhibitor is/are more effectively delivered to the mucosa of the gastrointestinal tract, such as the stomach or duodenum, by virtue of the water-swellable or water-soluble polymeric material, which may be instrumental in providing a prolonged or otherwise increased interaction of the fatty acid material the lipase inhibitor with target structures at/in the mucosa. The same may apply to the optional amino acid(s), vitamin(s) and/or micro-nutrient(s) if incorporated within the ingestible particles

Possibly, the water-swellable or water-soluble polymeric material prolongs the integrity of the particle after ingestion as compared to a lipid particle without the water-swellable or water-soluble polymeric material. Prolongation of particle integrity is the prolongation of time during incubation under in vivo or simulated in vivo conditions in which the majority (more than 50%) of particles do not decrease their volume or mass or melt into droplets. Particle integrity may be readily inferred by visual inspection by the naked eye or by means of a microscope or through imaging technology, including microscopic imaging, and subsequent computer-aided image processing. Prolonged integrity of the lipid-containing particle may result in more rapid gastric emptying of the particles and therefore more rapid interaction of particle-derived fatty acids or fatty acid esters with the intestinal mucosa. Prolonged integrity of the lipid-containing particle may also result in the delivery of fatty acids or fatty-acid esters to the more distal parts of the small intestine such as the jejunum or ileum.

In any case, the inventors have found that the oral administration of the particles of the invention to human subjects leads to a sensation of satiety, or increased satiety.

Additionally, the inventors have found that the ingestible particles as defined herein, and in particular oral combination products comprising or prepared from a plurality of the particles together with lipase inhibitors, are capable of effectively minimizing orlistat-induced gastro-intestinal problems such as steatorrhea. Without wishing to be bound by theory, it is currently believed that upon oral administration, the lipids provided with the ingestible particles are—during hydration and swelling of the particles after ingestion, exchanged against lipids, in particular undigested triglycerides (due to the presence of orlistat) provided by a consecutively ingested meal, so that during gastro-intestinal transit a stable emulsion gel is formed that is initially composed of emulsifying polymer, water and lipids provided with the ingestible particles according to the invention, and eventually, in the large intestine, is mainly composed of emulsifying polymer, water and undigested lipids provide with a consecutively ingested meal. Such stable emulsion gel present in the large intestine is capable of binding a large amount of lipid, particularly undigested triglyceride and prevent the occurrence of faecal liquefaction and fatty diarrhea.

Possibly, the water-swellable or water-soluble polymeric material provides the particle with mucoadhesive and/or emulsifying properties, in particular in combination with a prolonged integrity of the particle.

As used herein, an ingestible particle is a particle which is in principle suitable for oral ingestion, or oral administration. A particle which by virtue of its composition, size and morphology would be suitable as a food component or a component of a pharmaceutical composition for oral use is an example of an ingestible particle.

The particles have a diameter in the range from about 0.01 mm to about 10 mm, or from about 0.05 mm to about 3 mm, which means that they, or at least the majority of the particles, would normally pass through a sieve having an aperture or opening size of about 10 mm, or 3 mm, respectively, but not through a sieve having an aperture or opening size of about 0.01 mm, or 0.05 mm, respectively. Optionally, the particles may also have a diameter in the range from about 0.1 mm to about 2.5 mm, or from about 0.1 mm to about 2 mm, such as about 0.25±0.20 mm, about 0.5±0.25 mm, about 1.0±0.25 mm, about 1.5±0.25 mm, or about 2.0±0.25 mm, respectively. Within a composition comprising a plurality of particles according to the invention, these particle sizes should be interpreted to characterise the preferred mass median sieve diameters of the ingestible particles.

If the particles are to be swallowed as such, it is also preferred that they have a mass median sieve diameter in the range from about 0.1 mm to about 3 mm. Also preferred are mass median sieve diameters in the range from about 0.5 mm to about 3 mm, or from about 0.75 mm to about 2.5 mm, or from about 1 mm to about 2 mm. In other preferred embodiments, the mass median sieve diameter may be in the range from about 0.1 mm to about 0.4 mm, from about 0.2 mm to about 0.5 mm, or from about 0.2 mm to about 0.4 mm, respectively. This applies irrespective of whether the ingestible particles themselves contain the lipase inhibitor or not.

For the avoidance of doubt, these preferred particle sizes are intended as a general teaching and are applicable to all alternative embodiments of the pharmaceutical combination product of the invention with respect to the selection of the ingestible particles as well as e.g. components A, B, C, D and/or E, and all uses of the pharmaceutical combination products.

The water-swellable or water-soluble polymeric material in the ingestible particles of component A is a hydrophilic or amphiphilic polymeric material capable of dissolving or swelling in an aqueous environment. The material may comprise an emulsifying and/or a mucoadhesive compound or mixture of emulsifying and/or mucoadhesive compounds, or it may be capable of inducing emulsification and/or optionally mucoadhesiveness to the particle. If it is a mixture, it may also comprise one or more constituents which are themselves not water-swellable and/or emulsifying and/or mucoadhesive, as long as the mixture is water-swellable.

As used herein, swelling by water, or in an aqueous environment, typically means the volume increase of a solid body caused by an influx, or diffusion process of water accompanied by hydration, i.e. wetting and absorption of moisture. Swelling may e.g. may expressed by the swelling value in percent calculated as (wₛ−w_{d})/w_{d}×100 (with w_{d}=initial weight of dry component and wₛ=weight of swollen component). For the purposes of this study, swelling, or swelling capacity, is to be understood as the swelling behavior in vivo and should thus be evaluated under conditions mimicking those in vivo; e.g. by placing a fixed amount (w_{d}) of the polysaccharide in excess drinking water of 37° C.±2° C. for 4 hours, before removing excess water with the help of a filter and weighing the weight of swollen component (wₛ). The term ‘non-swelling’ as used herein shall refer to a swelling value of not more than 10%, preferably not more than 5%.

An emulsion as used herein is preferably of the type o/w, i.e. a stable mixture of an oily or lipidic phase (the dispersed phase) dispersed in an aqueous phase (the continuous phase). A polymer according to the invention may be hydrated and predominantly present in the aqueous phase and serve to stabilize the dispersed oil droplets. In the presence of such polymer, the emulsion may be stabilized to such an extent, that the viscosity is increased and the emulsion is not a pourable liquid but an emulsion gel. Viscosity or yield values of such emulsion gels may be measured by viscosimetry or other shear force measurement equipment.

The water-soluble polymeric material is a hydrophilic or amphiphilic polymer of a solubility in water of at least 1 mg/L.

Mucoadhesiveness is the capability of adhering to a mucosa, or mucosal membrane.

Various conventional methods are available to determine mucoadhesiveness, such as tensile strength measurements, ellipsometry, or rheological measurements (D. Ivarsson et al., Colloids Surf B Biointerfaces, vol. 92, pages 353-359, 2012). Even though these methods may not provide absolute values for mucoadhesiveness as such, they indicate the presence and relative magnitude of mucoadhesiveness of a material.

To determine mucoadhesiveness in the context of the invention, it is preferred that a modified falling liquid film method (described among other method in Mucoadhesive drug delivery systems, Carvalho F. C. et al., Brazilian Journal of Pharmaceutical Sciences 46 (2010)) is employed. According to the method, the selected mucous membrane (e.g. from pig stomach) is placed in a petri dish together with simulated gastric fluid at a controlled temperature of 37° C. The petri dish is placed on a table undergoing a tilting movement. Both tilting movement and volume of buffer are selected so that small waves of buffer continuously run over the surface of the mucous tissue. In the falling liquid film method, a similar agitation is achieved by pumping buffer over mucosal tissue tilted at a 45° angle. The amount of particles remaining on the mucous membrane after a specified time interval can be quantified by various methods. For instance, particles can be counted, optionally using a magnifying glass or microscope, or they may be collected and measured gravimetrically.

In the context of the invention, the water-swellable or water-soluble polymeric material should preferably have, or induce, sufficient mucoadhesive strength to cause attachment to a mucosal membrane upon contact with, and to cause the particle or a component thereof to stay attached for a period of time which is significantly longer than a material which is not mucoadhesive, such as a solid triglyceride or a lipophilic polymer, e.g. polytetrafluoroethylene. In a preferred embodiment, the water-swellable or water-soluble polymeric material comprises a mucoadhesive polymer. In particular, it may comprise at least one polymeric material selected from poly(carboxylates), chitosan, cellulose ethers, and xanthan gum.

In a further preferred embodiment, the water-swellable or water-soluble polymeric material is a plant fibre. In the context of the invention, a plant fibre includes selected individual components of plant fibres or derived therefrom, as well as their mixtures. For example, a suitable water-swellable or water-soluble polymeric material is psyllium seed husk, or psyllium seed husk fibres, also referred to as psyllium husk or simply psyllium. Psyllium seed husk are the seed coats of the seeds of Plantago ovata, also known as Desert Indian wheat or Blond Psyllium. A major component of psyllium seed husk is soluble but indigestible polysaccharide fibres which are highly swellable in water. Psyllium is known as a source of dietary fibre and as a mild laxative or stool softener.

If a poly(carboxylate) is used, this is preferably selected from poly(acrylic acid), poly(methacrylic acid), copolymers of acrylic and methacrylic acid, and poly(hydroxyethyl methacrylic acid), or from alginic acid (or a salt thereof, such as sodium alginate) or pectin or carboxymethylcellulose. The cellulose ether is preferably selected from hydroxyethyl cellulose, hydroxypropyl cellulose, hydroxypropyl methylcellulose, and methylcellulose. If an ionic polymer is used such as a poly(carboxylate) and/or a carboxymethylcellulose, this may be at least partially neutralised, preferably as sodium or potassium salt, most preferably as the sodium salt. Moreover, the polymeric material may be at least partially crosslinked.

In a further preferred embodiment, the mucoadhesive polymer is a copolymer of acrylic acid and methacrylic acid, or of acrylic or methacrylic acid and maleic acid. The copolymer may be crosslinked with small amounts of a polyalkenyl polyether. Such copolymers are highly hydrophilic and capable of absorbing large amounts of water which causes their swelling.

Particularly suitable for carrying out the invention are, for example, carbomers. Carbomers resins are high molecular weight, crosslinked acrylic acid-based polymers. Commercial versions of carbomers are sold as e.g. Carbopol®, Noveon®, Pemulen®, Polygel®, Synthalen®, Acritamer®, or Tego Carbomer®. Most of these brands include various carbomer grades.

For example, the Carbopol® polymer series encompasses homopolymers, copolymers, interpolymers as exemplified by Carbopol® Aqua SF-1 (acrylate copolymer, a lightly cross-linked acrylate copolymer), Carbopol® Aqua SF-2 (acrylate crosspolymer-4), Carbopol® Aqua CC (polyacrylate-1 crosspolymer), Carbopol® 934 (carbomer, acrylate homopolymer cross-linked with allyl ethers of sucrose), Carbopol® 940 (carbomer), Carbopol® 941 (carbomer), Carbopol® 971P (carbomer, lightly crosslinked with allyl pentaerythritol), Carbopol® 71G (a free-flowing granular form of Carbopol® 971P for use in direct compression formulations), Carbopol® 974P (carbomer, highly crosslinked), Carbopol® 980 (carbomer), Carbopol® 980 (carbomer), Carbopol® 981 (carbomer, allyl pentaerythritol crosslinked), Carbopol® 1342 (acrylates/C 10-30 alkyl acrylate crosspolymer, copolymer of acrylic acid and C10-C30 alkyl acrylate crosslinked with allyl pentaerythritol), Carbopol® 1382 (acrylates/C10-30 alkyl acrylate crosspolymer, copolymer of acrylic acid and C10-C30 alkyl acrylate crosslinked with allyl pentaerythritol), Carbopol® 2984 (carbomer), Carbopol® 5984 (carbomer), Carbopol® Ultrez 10 (carbomer), Carbopol® Ultrez 20 (acrylates/C10-30 alkyl acrylate crosspolymer), Carbopol® Ultrez 21 (acrylates/C10-30 alkyl acrylate crosspolymer), Carbopol® Ultrez 30 (carbomer), Carbopol® ETD 2001, Carbopol® ETD 2020 (acrylates/C10-30 alkyl acrylate crosspolymer, interpolymer containing a block copolymer of polyethylene glycol and a long chain alkyl acid ester), Carbopol® ETD 2050 (carbomer).

Polymer grades approved for pharmaceutical use are preferred among these, such as those which comply with a pharmacopoeial monograph, such as the monograph “Carbomer” of the European Pharmacopoeia (Ph. Eur. 8) or the monographs in the US Pharmacopoeia/National Formulary (USP-NF) with the titles, “Carbomer 910”, “Carbomer 934”, “Carbomer 934P”, “Carbomer 940”, “Carbomer 941”, “Carbomer Homopolymer”, “Carbomer Copolymer”, “Carbomer Interpolymer”, or “Carbomer 1342”.

In a specific embodiment of the invention, the water-swellable or water-soluble polymeric material comprises polyacrylic acid; e.g. Carbopol® 971 P NF.

Also particularly suitable are polycarbophils (USP-NF), which represent high molecular weight acrylic acid polymers crosslinked with divinyl glycol. They provide excellent bioadhesive properties. An example of a preferred grade of polycarbophil is NOVEON® AA-1.

Optionally, the water-swellable or water-soluble polymeric material comprises at least one polysaccharide approved for oral use as excipient or food additive or food ingredient. The at least one polysaccharide may be selected from the groups of cationic polysaccharides, anionic polysaccharides and non-ionic polysaccharides.

Suitable cationic polysaccharides include, but are not limited to, chitosan, polysaccharides modified by means of quaternary ammonium groups (for example cationic guar gum, cationic cellulose, cationic hydroxyethyl cellulose, and cationic starch), derivatives thereof, or mixtures of two or more thereof.

Alternatively, the cationic polysaccharide is a polymeric material with basic amino groups which are at least partially protonated in a neutral environment. The cationic polysaccharide may be provided or incorporated as a free base, as a quantitatively protonated salt form, or any mixture of the two forms.

The “free base” form refers to a polymer such as polyglucosamine (chitosan) comprising amino side chains in the base form, e.g. —NH₂. The “salt form” refers to a polymer such as polyglucosamine (chitosan) comprising amino side chains in the salt form, e.g. —NH₃+Cl⁻ for chloride salts of ammonium groups. It is understood that the salt form may refer to mixtures of salts, e.g. the salt form may be composed of mixtures of different salts such as —NH₃+Cl⁻ and —NH₃+CH₃—COO⁻. “Any mixture of the two forms” refers to a polymeric material comprising amino groups, where a fraction of the amino groups is present in the free base form, e.g. as —NH₂ for primary amino groups, and a fraction of those side chains is present in the salt form, e. g. —NH₃+Cl⁻. For instance, such a mixture may be referred to as partial chloride salt of chitosan.

“Chitosan” for the purpose of the invention is defined as chitosan derived by deacetylation of chitin, which may be obtained e.g. from fungi or crustaceans, wherein the average degree of deacetylation is preferably more than about 75%, more than about 80%, more than about 90%, or more than about 95%, respectively. The degree of deacetylation refers to the percentage of the chitin's amino groups that are deacetylated. A particularly preferred chitosan is derived from fungal biomass selected from the group consisting of Candida Guillermondii, Aspergillus niger, Aspergillus terreus, and combinations thereof, the chitosan containing material having greater than 85 percent deacetylation of N-acetyl groups in the chitin and exhibiting a viscosity of less than 25 centipoise at 25° C. in 1 percent aqueous acetic acid.

Suitable anionic polysaccharides include, but are not limited to, sulphated glycosamino glycans including heparans, heparansulfates, heparins; alginates; propylene glycol alginates; carrageenans; cellulose sulfate; carboxymethyl cellulose; fucoidan; galactans containing glucuronic acid or galacturonic acid; chondroitins or chondroitin sulphates; gellan gums; hyaluronans and hyaluronic acids; modified starches such as octenyl succinate starches or monostarch phosphates, oxidized starches or carboxymethylated starches; pectic acids, pectins including amidated pectins, homogalacturonans, substituted galacturonans, rhamnogalacturonans, their methyl and ethyl esters; porphyrans; sulphated galactanes; tragacanth or gum karaya; xanthan gums and xylans.

One particularly suitable polycarboxylate polysaccharide is alginic acid. Alginic acid is a linear copolymer with homopolymeric blocks of (1-4)-linked β-D-mannuronate (M) and its C-5 epimer α-L-guluronate (G) residues, respectively, covalently linked together in different sequences or blocks. The monomers can appear in homopolymeric blocks of consecutive G-residues (G-blocks), consecutive M-residues (M-blocks) or alternating M and G-residues (MG-blocks).

The anionic polysaccharide may be incorporated in the form of a free acid, or as the neutralised salt form of the acid, or as a mixture of these, i.e. as a partially neutralised salt. The “free acid” form refers to a polymeric material comprising acid groups in the non-ionised, protonated acid form, e. g. —COOH or —SO₄ Hz. The “salt form” refers to a polymeric material with acid groups in the ionised form, or salt form, e. g. —COO⁻Na⁺ for sodium salts of carboxylates or —SO₄²⁻2Na⁺ for sodium salts of sulphates. It is understood that the salt form may refer to mixtures of salts, e.g. the salt form may be composed of mixtures of —COO⁻ Na⁺ and —COO⁻ K+ or —COO—Ca²⁺—COO⁻ salts. “Any mixture of the two forms” refers to a polymeric material comprising acid groups, where a fraction of those groups is present in the non-ionised acid form, e. g. as —COOH for carboxylic acids, and another fraction of the acid groups is present in the ionised salt form, e. g. —COO⁻Na⁺ for sodium salts of carboxylic acids. For instance, such a mixture may be referred to as partial sodium salt of alginic acid.

Preferably, the anionic polysaccharide is an anionic dietary fibre. Dietary fibres, for the purpose of the invention, are carbohydrate polymers with ten or more monomeric units which are not hydrolysable by endogenous enzymes in the small intestine of humans. They typically represent carbohydrate polymers which have been obtained from food raw material by physical, enzymatic or chemical means, or synthetic carbohydrate polymers.

Preferably, the at least one anionic polysaccharide is alginic acid, carboxymethylcellulose, hyaluronan, sodium alginate, propylene glycol alginate, carrageenan, gellan gum, pectin, tragacanth or xanthan gum. Particularly preferred is that the at least one anionic polysaccharide is carboxymethylcellulose, sodium alginate or propylene glycol alginate, pectin, xanthan gum, or hyaluronan. Preferably, a combination of anionic polysaccharides is employed, such as sodium alginate and xanthan, or, even more preferably, sodium alginate and pectin.

Pectic polysaccharides (pectins) are rich in galacturonic acid. Several distinct polysaccharides have been identified and characterised within the pectic group. Homogalacturonans are linear chains of α-(1-4)-linked D-galacturonic acid. Substituted galacturonans are characterized by the presence of saccharide appendant residues (such as D-xylose or D-apiose in the respective cases of xylogalacturonan and apiogalacturonan) branching from a backbone of D-galacturonic acid residues. Rhamnogalacturonan I pectins (RG-I) contain a backbone of the repeating disaccharide: 4)-α-D-galacturonic acid-(1,2)-α-L-rhamnose-(1). From many of the rhamnose residues, sidechains of various neutral sugars may branch off. The neutral sugars are mainly D-galactose, L-arabinose and D-xylose, with the types and proportions of neutral sugars varying with the origin of pectin. Another structural type of pectin is rhamnogalacturonan II (RG-II). Isolated pectin has a molecular weight of typically 60-130,000 g/mol, varying with origin and extraction conditions. In nature, around 80 percent of carboxyl groups of galacturonic acid are esterified with methanol. This proportion is decreased to a varying degree during pectin extraction. The ratio of esterified to non-esterified galacturonic acid determines the behaviour of pectin in food applications. This is why pectins are classified as high- vs. low-ester pectins (short HM vs. LM-pectins), with more or less than half of all the galacturonic acid esterified. The non-esterified galacturonic acid units can be either free acids (carboxyl groups) or salts with sodium, potassium, or calcium. The salts of partially esterified pectins are called pectinates; if the degree of esterification is below 5 percent the salts are called pectates; the insoluble acid form, pectic acid. Amidated pectin is a modified form of pectin. Here, some of the galacturonic acid is converted with ammonia to carboxylic acid amide. Most preferred pectins are high ester pectins.

Suitable non-ionic polysaccharides include, but are not limited to, agaroses; amylopectins; amyloses; arabinoxylans; beta glucans including callose, curdlan, chrysolaminarin or leucosin, laminarin, lentinan, lichenin, pleuran, schizophyllan, zymosan; capsulans; celluloses including hemicelluloses, cellulose esters such as cellulose acetate, cellulose triacetate, cellulose propionate, cellulose acetate propionate and cellulose acetate butyrate; cellulose ethers such as methylcellulose, hydroxyethyl methylcellulose, hydroxypropyl methylcellulose, hydroxyethyl cellulose, hydroxypropyl cellulose, hydroxyethyl hydroxypropyl cellulose, methyl ethyl cellulose or alkoxy hydroxyethyl hydroxypropyl cellulose, wherein the alkoxy group is unbranched or branched and comprises 2 to 8 carbon atoms; chitins; cyclodextrins; dextrans; dextrins (for example commercially available as Nutriose®); galactoglucomannans; galactomannans including fenugreek gum, guar gum, tara gum, locust bean gum or carob gum; glucomannans including konjac gum; fructans including inulin, levan, sinistrin or phlein; maltodextrins; glycogens; pullulans; starches including resistant starches, modified starches such as acetylated starch, hydroxypropylated starch or hydroxyethyl starch; polydextroses; welan gum and xyloglycans.

Preferably, the non-ionic polysaccharide is a non-ionic dietary fibre. Preferably, the non-ionic polysaccharide is selected from the group consisting of beta glucans, cellulose ethers, guar gums, galactomannans, glucomannans, inulins and dextrins. Preferably, the non-ionic polysaccharide is hydroxypropyl methylcellulose or locust bean gum, or oat or barley beta glucan or konjac gum or resistant dextrin. Among the particularly preferred non-ionic polysaccharides is hydroxypropyl methylcellulose, hydroxypropylcellulose, and beta glucan from oat or barley and resistant dextrin from starch.

Resistant dextrins are partially hydrolysed starches; i.e. short chain glucose polymers, without sweet taste which are water-soluble and relatively resistant to the hydrolytic action of human digestive enzymes. They can be made for instance from either wheat (Nutriose® FB range or Benefiber®) or maize starch (Nutriose® FM range), using a highly controlled process of dextrinisation (heating the starch in the presence of small amounts of food-grade acid), followed by a chromatographic fractionation step. This produces a highly indigestible, water-soluble dextrin, with a high fibre content of about 65-85%, and a more narrow, favourable molecular weight distribution; e.g. approx. 4000 to 6000 Da for Nutriose® 6, or 3500 to 4500 Da for Nutriose® 10 (other dextrins, e.g. one of the starting materials to prepare resistant dextrins, may exhibit broader molecular ranges such as from about 3000 to 10,000 Da). During the dextrinisation step, the starch undergoes a degree of hydrolysis followed by repolymerisation that converts it into fibre and results in a drastically reduced molecular weight and the introduction of new glucoside linkages: in addition to the digestible starch α-1,4 and α-1,6 glycosidic linkages as commonly found in starches and the digestible maltodextrins, also non-digestible glycosidic bonds such as β-1,2 or β-1,3, are formed in resistant dextrins, which cannot be cleaved by enzymes in the digestive tract. As a result, a portion of the dextrin is not digested in the upper part of the gastro-intestinal tract and is not directly available as such for energy utilisation. Further, some commercial suppliers offer grades with different levels of mono- and di-saccharides (e.g. Nutriose® 10>Nutriose® 6, as available e.g. from Roquette), while the composition of the higher molecular weight oligomers is the same in both grades.

Optionally, the water-swellable or water-soluble polymeric material according to the invention comprises more than one polysaccharide. Preferred is in particular the selection of an anionic polysaccharide and a non-ionic polysaccharide, especially the combination of xanthan gum and hydroxypropyl methylcellulose.

Optionally, the water-swellable or water-soluble polymeric material according to the invention comprises a synthetic water-swellable or water-soluble polymeric material such as polyvinyl alcohol, polyvinyl acetate, polyethylene glycols (PEG), polypropylene glycols (PPG) or polyvinylpyrrolidones (PVP). Such polymer may be linear, branched or crosslinked, as for instance in crospovidone (crosslinked polyvinylpyrrolidone), or a PEG hydrogel.

Optionally, the water-swellable or water-soluble polymeric material comprises a thiolated polymer such as chitosan-4-thiobutylamidine, a chitosan-thioglycolic acid conjugate, a chitosan-cysteine conjugate, a chitosan glutathione conjugate, a polycarbophil-cysteine conjugate, a polyacrylic acid-cysteine conjugate, a carboxymethyl cellulose-cysteine conjugate, or any mixture or combination of two or more of these.

The first lipid material of the ingestible particles comprises a medium or long chain fatty acid compound. A fatty acid compound, as used herein, may also refer to a free fatty acid, a partially or completely neutralised fatty acid, i.e. the salt of a fatty acid, such as a sodium, potassium or calcium salt, or an esterified fatty acid. An esterified fatty acid may have, as alcohol residue, a glycerol, so that the esterified fatty acid is a mono-, di- or triglyceride. The acyl chain of the fatty acid may be saturated or unsaturated. In an optional embodiment, first lipid material may comprise none or only a small amount of di- and triglyceride; e.g. the content of di- and triglycerides within the first lipid material may be 80% or less, or even 50% or less. In a further optional embodiment, the fatty acid compound is a fatty acid mono ester, e.g. an ethyl ester. These provisions may help to further prevent, or at least limit, faecal liquefaction under orlistat treatment when lipase activity is blocked; in particular for di- and triglycerides exhibiting low melting ranges (below 37° C.), since the triglycerides will not be hydrolysed into absorbable free fatty acids and would then remain as a molten liquid inside the gut lumen until being excreted.

A medium chain fatty acid is understood as fatty acid with an acyl residue of 6 to 12 carbon atoms, whereas a long chain fatty acid means a fatty acid with an acyl chain of 13 to 21 carbon atoms. Among the preferred medium chain fatty acids are caprylic acid, capric acid, and lauric acid, including their esters and salts, in particular their mono-, di- and triglycerides and their sodium, potassium and calcium salts. In the case of di- and triglycerides, these may also have different fatty acid residues per glyceride molecule. Examples of preferred long chain fatty acids include myristic acid, palmitic acid, stearic acid, arachidic acid, behenic acid, myristoleic acid, palmitoleic acid, sapienic acid, oleic acid, linoleic acid, and linolenic acid, and the respective salts and glycerides.

In one of the preferred embodiments, the first lipid material comprises one or more partial glycerides of a medium or long chain fatty acid, in particular monoglycerides of a medium or long chain fatty acid. For example, monoolein or monolaurin are very suitable for carrying out the invention, individually or in combination with each other. As used herein, a monoglyceride such as monoolein or monolaurin may be incorporated as a substantially pure compound or as a mixture of mono- and diglycerides or even mono-, di- and triglycerides with various fatty acids, but with a high content (“enriched”) of a particular monoglyceride compound. For example, a monoolein grade may be used which comprises at least about 40% (or at least about 50%, or 60% or 70% or 80% or 90%) of the actual monoglyceride of oleic acid.

The first lipid material may of course represent a mixture incorporating two or more fatty acids, and/or fatty acid esters or salts. For example, the component may comprise one or more a fatty acids, which may be partially or completely neutralised, in combination with one or more glycerides, such as triglycerides.

The constituent(s) of the first lipid material may represent a native, synthetic or semisynthetic material.

In one embodiment, the first lipid material comprises one or more free fatty acids. For example free oleic acid or lauric acid may be part of the lipid material. Other preferred free fatty acids are mixtures of unsaturated fatty acids such as the so-called omega fatty acids or conjugated linoleic acids. Conjugated linoleic acids (CLA) are a family of isomers of linoleic acid. Conjugated linoleic acid is both a trans fatty acid and a cis fatty acid as the double bonds of CLAs are conjugated and separated by a single bond between them. Brands of CLAs are marketed as dietary supplements (Tonalin, BASF, and Clarinol, Stepan). Omega-3 fatty acids are polyunsaturated fatty acids (PUFAs) with a double bond (C═C) at the third carbon atom from the end of the carbon chain. Examples for omega-3 fatty acids are α-linolenic acid (ALA) (found in plant oils), eicosapentaenoic acid (EPA), and docosahexaenoic acid (DHA) (both commonly found in marine oils). If the first lipid material comprises an unsaturated fatty acid, it may also comprise an antioxidant such as vitamin E or a derivative thereof.

In one embodiment, the medium or long chain fatty acid compound in the first lipid material, either per se in vitro or in the hydrated state in vivo, has a melting range of below 37° C. As used herein, the melting range is understood as being below 37° C. if the lower (but not necessarily the upper) limit of the range is below 37° C. In other words, a compound having a melting range of 35° C. to 38° C. is an example of a material with a melting range of below 37° C. according to the invention. In other words, at least some of the fatty acid material in the lipid material should melt at the physiological temperature of the human body according to this embodiment. Moreover, the specified melting range is also met if the lipid material is capable of hydration, wherein the melting range in the hydrated state is below 37° C. Such behaviour of some lipids has also been described as “melting by hydration”. In one of the preferred embodiments, the melting range refers to the fatty acid glyceride component as such, i.e. not in its hydrated state.

According to a further preference, the first lipid material comprises a medium or long chain fatty acid compound having a melting range, or lower limit of the melting range, between about 10° C. and 37° C., or between about 25° C. and 37° C., respectively.

It has been surprisingly found by the inventors that ingestible particles containing the water-swellable or water-soluble polymeric material embedded in, or coated with, a lipid material comprising such low-melting fatty acid compound(s) are capable of exhibiting a prolonged integrity of the particles. Possibly, mucoadhesive properties are inferred to the particles. Possibly, these effects alone or in combination also contribute to, or are related to, the prolonged gastric residence time of the particles, the increased bioavailability of lipid and the induction of satiety caused by their administration. The same may apply to the bioavailability of the lipase inhibitor and/or the amino acid(s) for the optional embodiments where the ingestible particles further comprise the lipase inhibitor and/or an amino acid (e.g. embedded within, or coated with, the lipid material along with the water-swellable or water-soluble polymeric material)

It has further surprisingly been found by the inventors that particles containing the water-swellable or water-soluble polymeric component embedded in, or coated with, a lipid component comprising such low-melting fatty acid compound(s) is capable of forming a viscous emulsion in the gastrointestinal tract. Possibly, this effect also contributes, or is related to, the prolonged gastric residence time of the particles and the induction of satiety caused by their administration.

Optionally, the first lipid material with a melting range below 37° C. may comprise one or more further constituents which may have entirely different melting ranges. For example, a mixture of oleic acid, which has a melting range of 13° C. to 14° C., and a hard fat (i.e. a mixture of triglycerides) having a melting range of 42° C. to 45° C. may be used as the first lipid material. As an alternative to the hard fat, myristic acid (mp 54° C. to 55° C.) or lauric acid (mp 43° C. to 44° C.) may be used in such mixture. It may also be advantageous to combine a fatty acid with the salt of a fatty acid at a selected ratio such as to adjust the melting range to a desired optimum.

Alternatively, and in one of the preferred embodiments, the fatty acid compound in the first lipid material, either per se in vitro or in the hydrated state in vivo, has a melting range of above 37° C. As used herein, the melting range is understood as being above 37° C. if the lower limit of the range is above 37° C. In other words, a compound having a melting range of 40° C. to 44° C. is an example of a material with a melting range of above 37° C. according to the invention. Moreover, the specified melting range is also met if the lipid material is capable of hydration, wherein the melting range in the hydrated state is still above 37° C. In one of the preferred embodiments, the melting range refers to the fatty acid glyceride component as such, i.e. not in its hydrated state. A particularly preferred first lipid material having a melting range of above 37° C. is fractionated but non-hydrogenated palm stearin or palm kernel stearin. Palm stearin is the solid fraction of palm oil that is produced by partial crystallization at controlled temperature. A particularly preferred commercial quality is Prifex® 300 from Sime Darby Unimills.

It has been surprisingly found by the inventors that ingestible particles containing the water-swellable or water-soluble polymeric material embedded in, or coated with, a lipid material comprising such higher-melting fatty acid compound(s) are also capable of exhibiting a prolonged integrity of the particles. Possibly, mucoadhesive properties are inferred to the particles. Possibly, these effects alone or in combination also contribute to, or are related to, the prolonged gastric residence time of the particles, the increased bioavailability of lipid and the induction of satiety caused by their administration. The same may apply to the bioavailability of the lipase inhibitor and/or the other optionally incorporated components (amino acid(s), vitamin(s) and/or micro-nutrient(s)) for the optional embodiments where the ingestible particles further comprise these components (e.g. embedded within, or coated with, the lipid material along with the water-swellable or water-soluble polymeric material).

In addition, the selection of fatty acid compounds in the first lipid material which have a melting range of above 37° C. will prevent, or at least limit, faecal liquefaction under orlistat treatment, when lipase activity is blocked, since e.g. the triglycerides will not be hydrolysed into absorbable free fatty acids yet remain solid until being excreted.

According to the invention, the water-swellable or water-soluble polymeric material of the ingestible particles is embedded within, and/or coated with, the lipid material. As used herein, the term ‘embedded’ means that the water-swellable or water-soluble polymeric material is largely dispersed within the lipid material, whether molecularly, colloidally or in the form of a solid suspension. The lipid material forms a continuous phase in which the water-swellable or water-soluble polymeric material is discontinuous and in dispersed form. For the avoidance of doubt, this does not exclude that some of the material representing the water-swellable or water-soluble polymeric material—typically a small fraction—is not fully embedded, but positioned at the outer surface of the lipid material.

Typically, ‘embedded’ also means in the context of the invention that the lipid material and water-swellable or water-soluble polymeric material are mixed so intimately that the porosity of the resulting lipid-polymer composition is greatly reduced as compared to the particles formed from the water-swellable or water-soluble polymer itself, for instance as formed by roller compaction or agglomeration. Particle porosity may be determined by porosimetry, an analytical technique used to determine various quantifiable aspects of a material's porous nature, such as pore diameter, total pore volume, and surface area. The technique involves the intrusion of a non-wetting liquid at high pressure into a material through the use of a porosimeter.

The term ‘coated’ as used herein means that a particle comprising water-swellable or water-soluble polymeric material—as well as the amino acid, vitamin, micro-nutrient and/or the lipase inhibitor if present—is substantially surrounded with a layer of the lipid material representing the first lipid material. In practice, both forms (‘embedded in’ or ‘coated with’) may co-exist to some degree, depending on the method of preparation.

The water-swellable or water-soluble polymeric material and the other components such as the amino acid(s), the vitamin(s), the micro-nutrient(s) and/or the lipase inhibitor may be incorporated within the particles of the invention in different ways. For example, hydrophilic compounds such as amino acid(s) may be incorporated in admixture with the water-swellable or water-soluble polymeric material, whereas lipophilic compounds such as the lipase inhibitor and/or lipophilic vitamins may be incorporated in admixture with the lipid material.

In one of the preferred embodiments, the particles of the invention may be designed to exhibit an active core and a coating covering the core, wherein the active core comprises the first lipid material with the embedded or coated water-swellable or water-soluble polymeric material, whereas the coating comprises a second lipid material and/or a hydrophilic material. The coating may be substantially free of the water-swellable or water-soluble polymeric material. As used herein, the term “substantially free” means that the coating contains less than a functional amount of the water-swellable or water-soluble polymeric material, typically less than 1 wt-%, preferably less than 0.1 wt-% or even 0.01 wt-%, and also including zero percent of the water-swellable or water-soluble polymeric material. I.e. the particle comprises the water-swellable or water-soluble polymeric component embedded in or coated with the first lipid component of the active core, with optional additions of the amino acid and/or the lipase inhibitor to the particle. Optionally, the latter two may also be embedded in or coated with the first lipid component of the active core.

This embodiment with an active core and a coating is particularly useful in that the coating allows for convenient oral administration without the water-swellable or water-soluble polymeric material interacting with the mucosa of the mouth or oesophagus during ingestion, as the coating acts as a protective layer. The same may apply to the amino acid, the vitamin, the micro-nutrient and/or the lipase inhibitor if present in the active core but not in the coating. The coating also provides protection against agglomeration and sintering during manufacture, storage and shipping, and contributes to achieving an acceptable shelf life.

In other words, in this group of embodiments, the active core may be coated which a physiologically inactive coating, such as a polymeric film coating or a lipid coating. The polymeric film coating, which is based on a hydrophilic material, may be free of lipid, or it may comprise some relatively small amount of lipid e.g. as plasticiser. The lipid coating may be solely composed of the second lipid material, or it may contain some amount of the hydrophilic material, e.g. as disintegration enhancer.

The coating may be designed to be rapidly disintegrating so that the active core of the particles is released rapidly after swallowing. Preferably, the second lipid material, i.e. that which is incorporated in the coating of the particles, comprises one or more lipids having a melting point or melting range below about 37° C., as defined above, such as a melting range between about 25° C. and about 37° C. The composition of the second lipid material may optionally be the same as that of the first lipid material. Alternatively, it may be different.

As said, the coating of the particle according to this embodiment may comprise a hydrophilic material. This hydrophilic material may be embedded or dispersed within the second lipid material and may act as a disintegration enhancer for the coating layer.

Disintegration enhancement may be achieved by various mechanisms, depending on the choice of the hydrophilic material. For example, a disintegrant—such as e.g. crospovidone, croscarmellose, low-substituted hypromellose or even ion-exchange resins may rapidly take up water, expand in volume and thereby cause the disruption of the coating. Non-swelling, highly water-soluble excipients such as sugars or sugar alcohols, on the other hand, may predominantly act as pore formers by which water channels are rapidly created by which disintegration is also enhanced. Optionally, the hydrophilic material comprises a mixture of hydrophilic compounds. Preferably, the hydrophilic material is different from the water-swellable or water-soluble polymeric material and has no or only a low degree of mucoadhesiveness.

Optionally, the hydrophilic component comprises the amino acid, or even consists of the amino acid. If the particle comprises more than one amino acid, the hydrophilic component may comprise, or consist of, one of the amino acids, or some of the amino acids, or all of the amino acids. Same applies to hydrophilic vitamins and/or hydrophilic micro-nutrients.

If the coating only contains the hydrophilic material but no lipid material, the hydrophilic material preferably represents a film-forming agent such as a water soluble polymer. Examples of potentially suitable film-forming polymers include methylcellulose, hyprolose, hypromellose, polyvinyl alcohol, povidone, polyvinyl acetate, (meth)acrylate copolymer, and the like. Optionally, the composition may comprise further ingredients such as one or more plasticisers, pH-modifying agents, pore formers, colouring agents, sweetening agents, flavours, anti-tack agents, or dispersion aids.

In this group of embodiments where the particles of the invention exhibit an active core comprising the first lipid material with the embedded or coated water-swellable or water-soluble polymeric material and being surrounded by a coating, it is furthermore preferred that the active core contributes at least about 50% to the weight of the total particles. Optionally, the weight of the active core is at least about 60%, or even at least about 70% of the total particle's weight.

Optionally, the particles exhibiting an active core may further comprise an amino acid, a vitamin, a micro-nutrient or any combination thereof, in addition to the first lipid material and the water-swellable or water-soluble polymeric material. In this case the water-swellable or water-soluble polymeric material and/or the amino acid is typically embedded within, and/or coated with, the lipid material forming the active core. Optionally, also the vitamin(s) and/or the micronutrient(s) may be embedded within, or coated with, the lipid material forming the active core.

For those specific embodiments of the invention, where the ingestible particles contain the lipase inhibitor, said inhibitor may be contained in the active core and/or the coating. For the above mentioned reasons (e.g. protection against agglomeration and sintering during manufacture, storage and shipping, and acceptable shelf life), though, it may be more advisable to incorporate the lipase inhibitor into the active core, along with the water-swellable or water-soluble polymeric material.

In a related embodiment, the particle according to the invention comprises an inert core, a first coating covering the inert core, and a second coating covering the first coating. In this case, the first coating comprises the water-swellable or water-soluble polymeric material and the first lipid material, the second coating comprises a second lipid material and optionally a hydrophilic material, and the second coating is also substantially free of the water-swellable or water-soluble polymeric material. The hydrophilic component may be selected as described above. As in the previously discussed embodiment, the first lipid material with the embedded or coated water-swellable or water-soluble polymeric material is surrounded with a coating layer comprising the second lipid material. The difference is that the first lipid material and the water-swellable or water-soluble polymeric material do not form the core of the particle, but a layer on an inert core having a different composition. The inert core may be composed of a pharmacologically inert material such as sucrose, starch or microcrystalline cellulose. Specific examples of suitable inert cores include spheroids with average diameters in the range of about 100 or 200 mm based on microcrystalline cellulose which are e.g. commercially available as Cellets® 100 or Cellets® 200; nonpareils of starch and sugar of similar diameter; or sugar crystals of similar diameter, e.g. as obtainable by sieving.

With respect to the composition and further optional features of the lipid materials, the water-swellable or water-soluble polymeric material, the amino acid, the vitamin, the micro-nutrient, the lipase inhibitor and the hydrophilic material, reference is made to the discussion above.

In the context of this embodiment, the inert core should preferably not contribute more than about 70% to the weight of the total particle. More preferably, the weight of the core is not higher than about 60%, or not higher than about 50% of the total particle weight. In other embodiments, the weight of the core is from about 10% to about 50%, or from about 10% to about 40%, or from about 15% to about 35% of the total particle weight.

For those specific embodiments of the invention, where the ingestible particles contain the lipase inhibitor, said inhibitor may be contained in the first coating and/or the second coating. For the above mentioned reasons (e.g. protection against agglomeration and sintering during manufacture, storage and shipping, and acceptable shelf life), though, it may be more advisable to incorporate the lipase inhibitor into the first coating, along with the water-swellable or water-soluble polymeric material.

Optionally, the particles exhibiting a first coating on an inert core may further comprise an amino acid, a vitamin, a micronutrient or any combination thereof, in addition to the first lipid material, the water-swellable or water-soluble polymeric material, and optionally the lipase inhibitor. In this case the water-swellable or water-soluble polymeric material and/or the amino acid is typically embedded within, and/or coated with, the lipid material forming the first layer. Optionally, also the vitamin(s) and/or the micronutrient(s) may be embedded within, or coated with, the lipid material forming the first layer.

As already discussed, it is a key feature of the invention that the water-swellable or water-soluble polymeric material—and optionally also the lipase inhibitor, an amino acid, vitamin and/or micro-nutrient—is embedded within, or coated by, the first lipid material, which appears to effect an improved and/or prolonged interaction of the fatty acid, and/or of the above described optional components, with their target structures at/in the gastrointestinal mucosa. A target structure may, for example, be represented by G-protein coupled receptors (GPCRs) involved in the sensing of intestinal lipids such as GPR120.

In some embodiments, this may also result in an increased bioavailability of the first lipid material (and optionally also of the lipase inhibitor, the amino acid, the vitamin and/or of the micro-nutrient). In this context, bioavailability should be broadly understood such as to include the availability of the first lipid material, or the biologically active constituents thereof, at a biological target site, such as the gastric or intestinal mucosa, in terms of the extent and/or duration of availability.

To further enhance this effect, it is preferred that the weight ratio of the first lipid material to the water-swellable or water-soluble polymeric material is in the range from about 0.1 to about 10. In some embodiments, the weight ratio is from about 0.1 to about 5, from about 0.1 to about 3, from about 0.1 to about 2, or from about 0.1 to about 1. In further embodiments, this weight ratio is from about 0.2 to about 1.5, from about 0.25 to about 1.2, from about 0.25 to about 1.0, such as about 0.3, about 0.5., about 0.75, or about 1, respectively. Particularly preferred is a weight ratio from about 0.5 to about 5, or from about 0.75 to about 4, or from about 1 to about 3, respectively. For the avoidance of doubt, these preferred ratios are intended as a general teaching and are applicable to all alternative embodiments of the pharmaceutical combination product of the invention with respect to the selection of the ingestible particles as well as e.g. components A, B, C, D and/or E, and apply to all uses of the pharmaceutical combination products.

As mentioned before, in optional embodiments of the invention, the particles may further comprise an amino acid, a vitamin, a micro-nutrient or any combination thereof; e.g. in addition to the first lipid material and the water-swellable or water-soluble polymeric material.

As used herein, an amino acid is a compound having an amino group and a carboxyl group. Optionally, the carboxylic group is partially or fully neutralised.

The particles preferably comprise one or more amino acids selected from proteogenic amino acids, i.e. amino acids which are potential precursors of a protein in that it may be incorporated into a protein during its translation, or biosynthesis. Proteogenic L-amino acids as currently identified are L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, L-valine, L-selenocysteine, L-pyrrolysine, and N-formyl-L-methionine.

In another embodiment, the amino acid is selected from the twenty amino acids which form the genetic code, which group consists of L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, and L-valine.

In another preferred embodiment, the amino acid is selected from the group of the so-called essential amino acids which consists of those amino acids which the human organism cannot synthesize, i.e. L-histidine, L-isoleucine, L-leucine, L-lysine, L-methionine, L-phenylalanine, L-threonine, L-tryptophan, and L-valine.

In a further preferred embodiment, the amino acid is selected from the group consisting of L-isoleucine, L-valine, L-tyrosine, L-methionine, L-lysine, L-arginine, L-cysteine, L-phenylalanine, L-glutamate, L-glutamine, L-leucine, and L-tryptophan.

From these, the group consisting of L-phenylalanine, L-leucine, L-glutamine, L-glutamate, and L-tryptophan is particularly preferred.

In another particularly preferred embodiment, the amino acid is L-tryptophan.

Optionally, the particles comprise two or more amino acids. Such mixture or combination of amino acids should preferably comprise at least one amino acid as described above, i.e. a proteogenic amino acid, or an amino acid from the group of amino acids forming the genetic code, or from the essential amino acids, or the group of amino acids consisting of L-isoleucine, L-valine, L-tyrosine, L-methionine, L-lysine, L-arginine, L-cysteine, L-phenylalanine, L-glutamate, L-glutamine, L-leucine, and L-tryptophan. Particularly preferred embodiments of the particles with mixtures or combinations of amino acids comprise at least one amino acid from the group consisting of L-phenylalanine, L-leucine, L-glutamine, L-glutamate, and L-tryptophan. In particular, L-tryptophan is a preferred constituent of a combination of two or more amino acids.

Also preferred are mixtures or combinations of amino acids in which at least two amino acids are members of one of the preferred groups as previously defined. Moreover, mixtures or combinations of amino acids may be used in the particles in which essentially all incorporated amino acids are members of one of the preferred groups as previously defined.

As used herein, vitamins are organic compounds, or a related set of compounds, acting as vital nutrients required in small amounts, which e.g. humans (or other organisms) typically cannot synthesise in sufficient quantities and which therefore must be taken up with the diet. Their lack typically results in a pathological deficiency condition. The term ‘vitamin’ is conditional in that it depends on the particular organism; for instance ascorbic acid is a vitamin for humans, while many other animals can synthesise it. Vitamins are organic compounds classified by their biological and chemical activity, not by their structure. Each vitamin refers to a number of vitamers, all having the biological activity of the particular vitamin, convertible to the active form of the vitamin in the body, and grouped together under alphabetised generic descriptors, such as ‘vitamin A’. Universally recognised vitamins are preferred for the present invention (related vitamer(s) in brackets): vitamin A (retinol, retinal, and the carotenoids, including beta carotene, cryptoxanthin, lutein, lycopene, zeaxanthin), vitamin B1 (thiamine), vitamin B2 (riboflavin), vitamin B3 (niacin, niacinamide), vitamin B5 (pantothenic acid), vitamin B6 (pyridoxine, pyridoxamine, pyridoxal), vitamin B7 (biotin), vitamin B8 (ergadenylic acid), vitamin B9 (folic acid, folinic acid), vitamin B12 (cyanocobalamin, hydroxycobalamin, methylcobalamin), vitamin C (ascorbic acid), vitamin D (cholecalciferol (D3), ergocalciferol (D2)), vitamin E (tocopherols, tocotrienols), vitamin K (phylloquinone, menaquinones). The vitamins according to the invention may be provided as semisynthetic and synthetic-source supplements and/or as supplements of natural origin; such as in the form of plant extracts.

As used herein, the term ‘micro-nutrients’ refers to nutrients required by humans and/or other organisms in small quantities for a variety of their physiological functions, their proper growth and development; including, for instance, dietary micro-minerals or trace elements in amounts generally less than 100 mg/day (as opposed to macro-minerals). The micro-minerals or trace elements include at least boron, bromine, cobalt, chromium, copper, fluoride, iodine, iron, manganese, molybdenum, selenium, zinc. They may optionally be present in ionised or complexed form or as a salt, an oxide or a chelated salt.

Micro-nutrients also include phytochemicals, such as terpenoids or polyphenolic compounds, organic acids, choline, cholesterol as well as vitamins (i.e. some compounds may qualify for both categories, vitamins and micro-nutrients).

Preferred micro-nutrients according to the invention may be selected from organic acids, such as acetic acid, citric acid, lactic acid, malic acid, and taurine; and trace- or micro-minerals such as salts of boron, bromine, cobalt, chromium, copper, fluoride, iodine, iron, manganese, molybdenum, selenium, or zinc; choline and cholesterol.

The ingestible particles according to the invention (i.e. those containing a lipase inhibitor, and optionally an amino acid, a vitamin and/or a micro-nutrient; and/or those provided free of a lipase inhibitor but in the inventive pharmaceutical combination product together with an “extragranular” lipase inhibitor) may be provided in the form of granules, pellets, or minitablets. More preferably, the particles are provided in the form of granules and/or pellets. However, it should be noted, that the satiety inducing effect of the particles of the invention usually does not rely on the specific shape of the particle but on the particle's composition.

As used herein, a granule refers to an agglomerated particle which has been prepared from a plurality of smaller, primary particles. Hence, as used herein the term granule(s) does not necessarily imply a specific shape, since the final shape of the granule(s) will be guided by the specific method of preparation. Agglomeration, or granulation, for the purpose of preparing a granule, may involve the use of a dry, wet or melt granulation technique.

A pellet, as used herein, is understood as a particle with a relatively spherical or spheroidal shape. If prepared by an agglomeration process, a pellet is a special type of granule. However, pellets (i.e. spherical or spheroidal particles) may also be prepared by other processes than agglomeration. For the avoidance of doubt, the degree of sphericity of a pellet may differ in various technical fields. In the context of the invention, the sphericity of a pellet is in the typical range of pellets used in pharmaceutical formulations for oral use, which often have an aspect ratio of longest space diagonal divided by shortest space diagonal in the range of about 1 to 1.5.

A minitablet, often also referred to as a microtablet, is a unit formed by the compression or compaction of a powder or of granules. Typically, the compression is done on tablet presses using punches.

Minitablets, tablets or capsules comprising the ingestible particles of the invention are preferably formulated and processed in such a way that they rapidly disintegrate after oral administration. As used herein, disintegration is understood as a substantial physical change to the minitablet, tablet or capsule morphology, such as the rupture or detachment of the tablet's coating, the dissolution of a capsule or the disintegration of a tablet or minitablet to release particles or pellets or granules of the invention. For the detection of such tablet, minitablet or capsule disintegration behaviour, a microscope may be used. With respect to the apparatus, the hydrodynamic conditions, and the temperature, the method <701> of the United States Pharmacopeia 29 (USP29) may be used, except that water may be used as test medium and that the wire mesh may be adapted with respect to the mesh size or aperture to take the sieve diameter of the tablet, minitablet or capsule into account. When tested according to this method, the minitablets or tablets or capsules comprising particles according to the invention preferably disintegrate within not more than about 15 minutes. More preferably, they disintegrate within about 10 minutes or less. According to another embodiment, they disintegrate within about 8 minutes or less, or within about 5 minutes or less, respectively.

Particles according to the invention may be prepared by a method comprising a step of processing a mixture comprising the first lipid material and the water-swellable or water-soluble polymeric material—and optionally further components such as the lipase inhibitor, an amino acid, a vitamin, a micro-nutrient—by (a) extruding the mixture using a screw extruder; (b) spray congealing the mixture, optionally using a jet-break-up technique; (c) melt granulating the mixture; (d) compressing the mixture into minitablets; (e) melt injection of the mixture into a liquid medium; or (f) spray coating of the mixture onto inert cores.

Where the lipase inhibitor is supposed to be contained in the ingestible particles (rather than the lipase inhibitor being added separately to the pharmaceutical combination product of the invention), said lipase inhibitor may be co-processed along with the water swellable or water soluble polymeric material, using the above described method(s). Like this, the lipase inhibitor would also be embedded by and/or incorporated in the lipid material; and thus either form the active core or the first coating on an inert core. (Such localisation of the lipase inhibitor in the active core or the first coating on an inert core is not compulsory, though, as described earlier.)

Hence, in a further aspect, the invention provides ingestible particles having a sieve diameter in the range from 0.01 mm to 10 mm, or from 0.05 mm to 3 mm, said particle comprising (a) a water-swellable or water-soluble polymeric material, (b) a first lipid material; and (c) a lipase inhibitor such as orlistat and optionally (d) an amino acid, a vitamin, a micro-nutrient, or any combinations thereof as defined above, wherein the first lipid material comprises a medium or long chain fatty acid compound, and the water-swellable or water-soluble polymeric material is embedded within, and/or coated with, the lipid material. In this embodiment the ingestible particles themselves represent the pharmaceutical combination product in that the lipase inhibitor is already contained. Further lipase inhibitor additions (in form of “extragranular” lipase inhibitor and/or lipase inhibitor provided as part of a kit together with the ingestible particles) are still possible, though not necessary.

In a specific embodiment of the invention, the water-swellable or water-soluble polymeric material comprises polyacrylic acid; e.g. Carbopol® 971 P NF.

The preparation of the mixture comprising the first lipid material and the water-swellable or water-soluble polymeric material (and optionally the lipase inhibitor, an amino acid, a vitamin, and/or a micro-nutrient if present in the ingestible particles) may be accomplished by conventional means such as blending or high-shear mixing.

Optionally, the mixture is prepared using the same equipment which is also utilised for the subsequent step in which the particles are formed. For example, for preparing a melt to be used for melt congealing, melt granulation or melt injection, it may not be required to prepare a dry premix prior to melting the constituents of the melt, but the mixing and melting can be performed simultaneously in one step. Therefore, the mixture to be processed according to steps (a) to (f) above should be broadly interpreted to cover any form of combining the materials required for preparing the particles.

In one embodiment, the mixture is extruded using a screw extruder. Optionally, a twin-screw extruder is used for carrying out the extrusion step. The extruder should have a screen with an aperture that is useful for producing an extrudate with appropriate diameter, such as 0.5 mm or 1.0 mm. The screw speed may be selected in consideration of the capability of the extruder and on the processability of the mixture. For example, it may be useful to select a screw speed in the range from about 20 rpm to about 100 rpm.

Preferably, the extrusion step is carried out without the use of a solvent and at a relatively low temperature, such as below about 35° C., or below about 30° C., e.g. at room temperature. It is also preferred that the extrusion step is carried out at a temperature which is lower than the lower limit of the melting range of the lowest-melting constituent of the mixture, e.g. 20° C. below the melting temperature. This prevents leakage from the extruder as well as improving the mixing efficiency.

In one embodiment, the ingredients used for preparing the particles by extrusion are mixed or blended before they are fed to the extruder. Alternatively, the ingredients may be mixed using the same equipment which is utilised for the extrusion step. Thus, it is also preferred that the ingredients used for preparing extruded particles are provided to the extruder by co-feeding, using appropriate feeding equipment, and optionally recycled within the extruder (e.g. via internal bypass-loops) until a uniform, intimate mixture is obtained which is ready for subsequent extrusion.

Subsequent to the extrusion step, the extrudate may be spheronised in order to obtain approximately spherical particles. For this purpose, any conventional spheroniser may be used. The temperature of the spheroniser jacket should preferably be set to be lower than the lower limit of the melting range of the lowest-melting constituent of the mixture. The speed of the spheronisation plates may be set between about 200 rpm and about 2,000 rpm, such as about 500 rpm to about 1,500 rpm. Subsequent sieving may be performed in order to select an optimal particle size of the product.

In a particular embodiment, the particles are prepared from the mixture by spray congealing. This process may also be referred to as spray chilling or spray cooling. In this process, a liquid melt is atomised into a spray of fine droplets of approximately spherical shape inside a spray cooling chamber. Here, the droplets meet a stream of air or gas which is sufficiently cold to solidify the droplets. The air or gas stream may have a co-current, mix-current or counter-current direction of flow.

To improve the formation of droplets of appropriate size and shape, a heatable rotary spray nozzle or a fountain nozzle may be used. In the context of the invention, a high speed rotary nozzle is one of the preferred nozzle types for preparing the particles.

Optionally, the uniformity of the atomised droplets may be further enhanced by using a jet break-up technique, such as electrostatic droplet generation, jet-cutting, jet excitation or flow focusing. In general, jet break-up refers to the disintegration of a liquid/gas jet due to forces acting on the jet.

In electrostatic droplet formation processes, a nozzle equipped with an electrode is used which applies an electrical charge to the melt spray. In jet cutting, the spray is directed through a cutter similar to e.g. a rotary disc with apertures of defined size. Jet excitation means the excitation of the melt spray by ultrasonic waves, causing vibration and facilitating the separation of droplets.

Flow focusing results from combining hydrodynamic forces with a specific geometry, which may be achieved by using a pressure chamber pressurised with a continuous focusing fluid supply. Inside, a focused fluid is injected through a capillary feed tube whose extremity opens up in front of a small orifice linking the chamber with the exterior ambient. The focusing fluid stream moulds the fluid meniscus into a cusp giving rise to a microjet exiting the chamber through the orifice. Capillary instability breaks up the stationary jet into homogeneous droplets.

In another specific embodiment, the particles are prepared by injecting the melted mixture into a liquid. The liquid may be cooled to a temperature below room temperature, or preferably to substantially below the lower limit of the melting range of the lowest-melting constituent of the lipid material. The liquid should be selected taking the composition of the mixture into consideration, but also with an eye on safety and physiological tolerability. In many cases, ethanol is a suitable liquid.

In another embodiment, the particles may be formed by melt agglomeration, or melt granulation. In the context of the invention, agglomeration and granulation may be used interchangeably. For this purpose, the constituents of the mixture are mixed or blended and agglomerated, or granulated, in a suitable type of equipment, such as a heatable granulator, a high-shear mixer/granulator or a fluid bed granulator. Depending on the type of equipment, the granulation may be carried out by heating the mixture to a temperature at which at least one of its constituents softens or melts, under continuous stirring or mixing. In a conventional granulator, this may lead to larger agglomerates which are then passed through a sieve to obtain the desired particle size. If fluid bed equipment is used, the complete mixture may be fluidised and heated carefully up to the melting temperature of the lowest-melting constituent. Alternatively, the lowest-melting constituent may be melted and sprayed onto the fluidised powder mixture comprising the remaining constituents.

Optionally, the melt granules may be further processed and compressed into minitablets. For this purpose, it is preferred that the granules are first blended with one or more tablet fillers/binders to enhance the plasticity of the mixture. Moreover, conventional excipients to improve the flow of the granules and reduce their tackiness may also be added before compression. Tableting may be carried out using any conventional pharmaceutical tablet press, such as an eccentric press or a rotary press. Optionally, the press may be equipped with multi-punch tooling so that each compression yields a plurality of minitablets. Punches for very small tablet diameters are preferred for particles intended to be swallowed as such, such as between about 1 mm and about 3 mm, such as about 1.5 mm. For larger particles which are intended to be chewed, larger tablet diameters may be used, such as in the range from about 1 mm to about 10 mm.

Alternatively and depending the mixture's flow properties, the mixture of the first lipid material and the water-swellable or water-soluble polymeric material may also be compressed into mini-tablets as such; i.e. without a preceding melt granulation step.

In a further embodiment, the particles are prepared by spray coating the mixture comprising the first lipid material and the water-swellable or water-soluble polymeric material (and optionally the lipase inhibitor if present in the particles) onto inert cores. As used herein, an inert core is a particle from a physiologically acceptable material which is suitable for being coated, and which itself does not substantially contribute to the physiological effect of the particles of the invention, i.e. the induction of satiety. Examples of suitable cores include crystals of appropriate size and shape, such as sugar (sucrose) crystals. In one of the preferred embodiments, spherical beads or non-pareils made from sugar, starch, cellulose, in particular microcrystalline cellulose (e.g. Cellets®) are spray coated with the mixture.

The spray coating of the inert cores may, for example, be performed in a fluid bed apparatus. The mixture of the first lipid material and the water-swellable or water-soluble polymeric material may be melted and sprayed onto the fluidised core particles. Optionally, the amino acid, vitamin, and/or micro-nutrient, if present, may also be added to this melt. Further optionally, said mixture may also comprise the lipase inhibitor, such as orlistat Alternatively, an aqueous or organic dispersion (or suspension, which is understood as a sub-type of a dispersion) of the mixture is sprayed onto the fluidised cores in such a way that the water or solvent evaporates and the mixture of the first lipid material and the water-swellable or water-soluble polymeric material forms a coating on the inert core particles.

As in all other processes mentioned above, a subsequent step of classifying the resulting particles using a sieve in order to obtain a more uniform particle size distribution may be useful. Where necessary or useful, the particles may be dried at 25° C. under vacuum prior to classifying them.

For the preparation of particles according to the invention which further exhibit a coating (or second coating covering the first coating) comprising a second lipid material and/or a hydrophilic material but not the water-swellable or water-soluble polymeric material, such second coating may also be applied using conventional pharmaceutical spray coating techniques. In one of the preferred embodiments, fluid bed coating is used for this purpose, using particles according to the invention prepared as described above as active cores which are fluidised, and onto which either a melt or a dispersion/suspension of the second lipid material, or a solution or dispersion/suspension of the hydrophilic material is sprayed. If both the second lipid material and the hydrophilic material are present, they may be applied together in the form of a dispersion/suspension in water or solvent, or as a melt of the lipid in which the hydrophilic material is dispersed.

For the avoidance of doubt, these preferred preparation processes for the design and manufacture of the particles are intended as a general teaching and are applicable to all alternative embodiments of the pharmaceutical combination product of the invention with respect to the selection of the ingestible particles as well as e.g. components A, B, C, D and/or E, and apply to all uses of the pharmaceutical combination products.

According a further aspect of the invention, an ingestible particle is provided which is obtainable by the method(s) as described above.

Where provided separately from the ingestible particles, the lipase inhibitor may be provided in any dosage form suited to be used in the pharmaceutical combination product of the invention; preferably in the form of granules, pellets, minitablets, tablets, capsules and the like. Any inert excipients may be employed to the degree as they are required for formulating the lipase inhibitor into said dosage forms.

The specific dosage form chosen for the lipase inhibitor does not necessarily have to match that chosen for the ingestible particles. For instance, where the pharmaceutical combination product according to the invention is provided as a kit, the lipase inhibitor may be in the form of tablets or capsules, while the ingestible particles may e.g. be provided as pellets from a stick pack. Likewise, lipase inhibitor minitablets may be filled together with granules of the ingestible particles into a ready-to-use single dose stick pack.

However, where the lipase inhibitor and the ingestible particles are to be combined in e.g. a compressed tablet, their respective forms and size should match at least to the degree, that de-mixing phenomena during the tabletting step are avoided; e.g. by mixing lipase inhibitor pellets and pellets of the ingestible particles, both with similar size.

This means, that in a further aspect, the invention provides a solid composition for oral administration comprising a plurality of the particles as described above, or the pharmaceutical combination product according to the invention which has been prepared from a plurality of the particles, such as by compressing them into tablets. If not compressed into tablets, the particles may in principle be filled into capsules, sachets, stick packs, or containers (e.g. bottles or drink vials of glass or other materials). In one of the preferred embodiments, the particles, or granules, are filled into capsules, sachets, stick packs, bottles or containers in such a way that a single dose is accommodated in one primary package.

Optionally, the solid composition may comprise the particles along with one or more further inactive ingredients, such as e.g. colouring agents, stabilising agents, wetting agents, bulking agents, suspending agents, pH-modifiers, and/or flow-regulating agents.

The presentation and oral administration of the particles in the form of, or using, sachets, stick packs, bottles or containers is also useful as it is preferred that a relatively large amount of the solid composition is administered as a single dose. In one of the preferred embodiments, a single dose comprises at least about 2 g of the solid composition, preferably at least about 3 g thereof and more preferably at least about 5 g. In another embodiment, a single dose comprises from about 3 g to about 20 g of the solid composition. In further embodiments, the amount comprised in a single dose is from about 4 g to about 20 g or from about 4 g to about 15 g of the solid composition, or from about 5 g to about 15 g or from about 5 g to about 12 g, or from about 5 g to about 10 g, respectively.

Where the pharmaceutical combination product comprises further constituents, such as components A to E, the weight of a single dose will increase correspondingly of course. For instance, the amount of the pharmaceutical combination product representing a single dose may then be at least about 20 g or at least about 30 g, or at least about 40 g, or at least about 50 g, respectively; for example in the range from about 30 g to about 150 g, or from about 40 g to about 120 g, or from about 50 to about 100 g, respectively.

It should be understood that these weights refer to the single dose unit or package as provided, or sold, to the consumer; for instance excluding the weight of any liquids which are not present in the single dose unit or package during shipping and storage but which may be added directly prior to actual ingestion by the user, or consumer (like water, milk or juice being added to a single dose of particles in a bottle or drink vial package to form a drinkable suspension).

It should further be understood that the provision of single dose units or packages and their weights is not intended to exclude the option of multiple dose units or packages. The oral composition may also be provided in larger packages containing multiple doses together with instructions on obtaining a single dose; for instance a 350 g package containing a blend of any of components A to E with the particles of the invention with a serving suggestion printed on the side of the package, such as ‘Single serving about 70 g+200 mL added water’.

It is also preferred that the composition exhibits a high contents of the particles of the invention, such as at least about 50%, or at least about 60%, or at least about 70%, or at least about 80% by weight. Particularly preferred is a particle content in the composition of at least about 90%, or at least about 95%, or at least about 98%, such as about 100% by weight.

For the purpose of administration, the solid composition and/or the pharmaceutical combination product may be suspended in a liquid or semisolid vehicle. I.e. in a further aspect, the invention provides a liquid or semi-solid composition obtainable by dispersing the solid composition and/or the pharmaceutical combination product as defined above in an ingestible liquid. The liquid may simply be water or fruit juice or a dairy beverage such as milk or mixtures thereof. As used herein, the term milk comprises milk-varieties obtained from animals (e.g. cow-, goat- or sheep milk) as well as milk varieties of vegetable/plant origin (e.g. soy-, rice- or nut based milks). The ingestible liquid may optionally be provided together with the solid composition within a kit; e.g. both in separate primary packagings but distributed, or sold, in combination, such that the consumer, or user, himself/herself adds it to the solid phase directly prior to ingestion. This has the advantage that the nature and amount of liquid are controlled and the administration is more reproducible. Alternatively, the ingestible liquid may be provided in the same primary packaging as the ingestible particles, e.g. a drink vial or bottle, in the form of a ‘ready-to-use’ drink suspension, which does not require reconstitution by the consumer, or user, prior to ingestion. The reconstituted or ‘ready-to-use’ drink suspensions may have, for example, a volume in the range from about 30 mL to about 300 mL, or from about 50 mL to about 200 mL. In case that additional “extragranular” components, such as components A to E, are comprised in the pharmaceutical combination product, the amount of liquid used for reconstitution may be larger, such as from about 50 mL to about 500 mL.

In a preferred embodiment, the solid composition and/or the pharmaceutical combination product of the invention is administered as a suspension drink. It was found that the suspension drink of the invention is useful for administering large amounts, such as 1 g or more, and more typically at least 5 g, such as from about 10 g or more, of the solid composition and/or the pharmaceutical combination product while exhibiting good drinkability and mouth feel.

The amount of the first lipid material, which is a key ingredient of the composition, should preferably be at least about 1 g per single dose or per package. In another embodiment, a single dose comprises at least about 2 g of the first lipid material, such as about 3 g or about 4 g. In a further preferred embodiment, the content of the first lipid material per single dose is at least about 5 g.

A lipase inhibitor such as orlistat may be present in a single dose; e.g. by adding orlistat in dry powdered or granulated form to the lipid-containing ingestible particles for instance in a second filling step prior to sealing the dosage-containing object, such as a sachet, a stick pack or a mini bottle.

Alternatively, the lipase inhibitor may be incorporated within the lipid-containing particles and may be present in the lipid matrix formed by the first lipid material or may present in one of the particle coatings as described earlier. The particles containing lipase inhibitors may be prepared by the abovementioned methods, for instance by dispersing lipase inhibitor in at least one of the lipid materials.

As mentioned above, the pharmaceutical combination product may optionally comprise one or more components selected from A to E in addition to the lipase inhibitor and the ingestible particles which are provided separately from, or “extragranular” to, said particles, e.g. in the form of a flowable mixture of particles, such as a powder, a powder blend and/or a granulate. In one embodiment, one or more components selected from A to E are provided “extragranular” to the ingestible particles but in the same dosage form and/or primary packaging; e.g. in form of mixtures of the ingestible particles and powders and/or granulates of any one of the optional components A to E. Said mixtures may be compressed to tablets or filled into capsules, sachets, stick packs, vials, bottles, or containers. In one embodiment, a powder, a powder blend and/or a granulate of any one of the components A to E may be provided together with a plurality of the ingestible particles in one common stick pack or bottle, optionally also including the lipase inhibitor.

Alternatively, the component(s) selected from A to E may also be provided in a separate pharmaceutical composition and/or primary packaging, e.g. in the form of a kit together with the plurality of ingestible particles and the lipase inhibitor; i.e. in separate primary packagings but distributed, or sold, in combination.

The decision on how to add any one of the components A to E to the pharmaceutical combination product—e.g. whether in the same pharmaceutical composition as the particles or a separate one—is made independently for each component and may be guided e.g. by weight or -stability concerns as well as processability and/or dispersibility considerations.

### Component A

Component A comprises a native or modified protein. Preferably, component A comprises one or more proteins selected from vegetable protein and/or animal protein. The vegetable protein may be a legume protein, grain protein, nut protein, mushroom protein, and protein from the seeds of other plants, and the animal protein may, for example, be selected from milk protein, egg protein, and gelatin. Particularly suitable vegetable proteins include soy protein, rice protein, hemp seed protein, pea protein lupin protein and almond protein. Suitable milk proteins include in particular casein and whey protein. Suitable gelatins include gelatin from fish, cattle, pigs, or chicken.

In one embodiment, component A essentially consists of protein powder or a blend of two or more proteins. Alternatively, component A may comprise the protein or protein blend in granulated form, optionally along with one or more other substituents, such as a granulation aid.

In one embodiment, the pharmaceutical combination product of the invention comprises at least (i) a lipase inhibitor (ii) a plurality of ingestible particles as defined above and (iii) component A. In particular if the product is also used to substitute a meal, partially or entirely, it is preferred that component A is present. In this case, the amount of component A in the pharmaceutical combination product may be up to about 90 wt.-%, such as from about 5 wt.-% to about 75 wt.-%, or from about 8 wt.-% to about 60 wt.-%, or from about 10 wt.-% to about 50 wt.-%. In absolute terms, the amount of component A is preferably selected such that a single dose of the combination product comprises from about 3 g to about 50 g of protein, such as from about 5 g to about 30 g of protein, or from about 10 g to about 25 g of protein, respectively.

The ratio of the ingestible particles to component A may optionally be in the range from about 1:10 to about 5:1, or from about 1:5 to 2:1, respectively. The ratio of the first lipid material in the ingestible particles to the protein in component A may optionally be in the range from about 1:20 to about 3:1, such as from about 1:10 to about 1:1.

### Component B

Component B preferably comprises one or more dietary fibres selected from soluble and/or insoluble dietary fibres. The soluble dietary fibre is preferably a prebiotic or natural gum; and the insoluble fibre is preferably a cellulose, lichenin, chitin, hemicellulose, or lignin.

As used herein, a prebiotic is a compound or material that supports the growth of microorganisms that are hosted by a human and that are beneficial to the host. In particular, a compound or material that is a substrate for the gut microbiome of a human is an example of a prebiotic. Many but not all currently known prebiotics are fibres.

Suitable prebiotic fibres include for example resistant dextrins, inulin, galacto-oligosaccharides, mannan oligosaccharides, and gum arabic. Optionally, component B may comprise the prebiotic fibre in the form of a plant extract which is rich in such fibre, such as extracts from chicory root, asparagus, leek, Jerusalem artichoke, dandelion, garlic, garlic, onion, wheat bran, beans, oats, barley, or banana.

As used herein, a natural gum is a native or modified soluble polysaccharide, or polysaccharide-containing polymer, that substantially increases the viscosity when dissolved in an aqueous medium even at relatively low concentrations. Hence, soluble fibres may also be referred to as viscous fibres. The natural gum may be selected from the group of natural gums representing largely uncharged compounds, or from the group of charged gums, or polyelectrolytes.

Suitable uncharged natural gums may be derived from bacteria, such as xanthan gum, or from botanical sources, such as Psyllium seed husks, glucomannan, guar gum, beta glucans such as oat or barley beta-glucans, locust bean gum, chicle gum, mastic gum, tara gum, spruce gum or dammar gum. Suitable natural polyelectrolyte gums include for example gums from seaweeds, such as agar, alginic acids and alginates, carrageenan; or charged gums from bacteria, such as gellan gum; or from other botanical sources such as gum arabic, gum ghatti, gum tragacanth, pectin, or Karaya gum.

An insoluble fibre is understood as a fibre which is substantially insoluble in water at physiological pH and body temperature. Suitable insoluble fibres include non-starch polysaccharides such as cellulose, lichenin, chitin, hemicellulose, or lignin. Optionally, component B comprises such insoluble fibres in the form of a plant material or plant extract, such as wheat bran, corn bran, or fibre-enriched vegetable or fruit powders.

Component B may of course also comprise a mixture of different fibres, whether from the same or different categories.

If present in the combination product, component B may be incorporated at any suitable amount, and preferably at an amount of up to about 50 g per single dose of the combination product. Also preferred are amounts from about 0.5 g to about 40 g, or from about 1 g to about 30 g, or from about 2 g to about 25 g, respectively.

### Component C

Component C comprises a vitamin, a micro-nutrient such as one or more micro-minerals, organic acids, choline, cholesterol, and/or a further dietary element (also called mineral nutrients). The definitions of vitamins and micro-nutrients as provided above equally apply to component C. The selection of the number, type and/or combination of the one or more vitamins and/or micro-nutrients in component C may be identical to that of the vitamins and/or micro-nutrients optionally employed inside the ingestible particles as described above. However, this is not a requirement; i.e. the ingestible particles may also contain different vitamins and/or micro-nutrients than component C.

A dietary element, often also referred to as an essential element, dietary mineral or mineral nutrient, is a chemical element that is physiologically required by the human body. Dietary elements are sometimes classified in various groups. For example, one group consist of hydrogen, carbon, nitrogen and oxygen, and is considered the basis of life and the quantitative basis of most organic compounds that play a role in human physiology. Another group which consists of sodium, potassium, magnesium, calcium, phosphorus, sulphur, and chlorine is often termed the quantitative elements or macro-minerals, as these elements are physiologically required in substantial amounts. The remaining elements are referred to as micro-minerals (see above under micro-nutrients), trace elements, or essential trace elements, as the amount that is physiologically required is very small.

Preferably component C comprises one or more of the following:
a vitamin selected from retinol, retinal, beta carotene, thiamine, cyanocobalamine, hydroxycyanocobalamine, methylcobalamine, riboflavin, niacin, niacinamide, pantothenic acid, pyridoxine, pyridoxamine, pyridoxal, biotin, folic acid, folinic acid, ascorbic acid, cholecalciferol, ergocalciferol, tocopherol, tocotrienol, phylloquinone, and menaquinone;
a micro-mineral selected from boron, bromine, chromium, cobalt, copper, fluoride, iodine, iron, manganese, molybdenum, selenium and zinc (optionally in ionised or complexed form or as a salt, an oxide or a chelated salt);
an organic acid such as acetic acid, citric acid, lactic acid, malic acid, or taurine;
choline,
cholesterol, and/or
a further dietary element such as a macro-mineral selected from calcium, chlorine, magnesium, phosphorous, potassium, sodium and sulphur (optionally in ionised or complexed form or as a salt, an oxide or a chelated salt).

For micro-nutrients, vitamins and dietary elements, recommendations have been established with respect to the daily intake level that is considered sufficient, adequate and/or acceptable for an average healthy individual by various national and international agencies. For example, the Institute of Medicine of the National Academies of the United States has published a system of nutritional recommendations referred to as the Dietary Reference Intake (DRI), which includes amongst others the Estimated Average Requirement (EAR), expected to meet the nutritional needs of 50% of a specific target group; the Recommended Dietary Allowance (RDA), which is the daily nutrient intake that is considered sufficient for the vast majority (at least 97.5%) of healthy individuals in a specific sex and age group; and the Tolerable Upper Intake Levels (UL), reflecting a maximum daily intake level that appears to cause no harm. The currently recommended EAR, RDA and UL values for micro-nutrients, vitamins and dietary elements are listed in the table below.

| | | | |
|---|---|---|---|
| Nutrient | EAR | RDA | UL |

| | | | | | | |
|---|---|---|---|---|---|---|
| Calcium | 800 | mg | 1000 | mg | 2500 | mg |

| | | | | | |
|---|---|---|---|---|---|
| Chloride | NE | 2300 | mg | 3600 | mg |

| | | | | |
|---|---|---|---|---|
| Chromium | NE | 35 | μg | ND |

| | | | | | | |
|---|---|---|---|---|---|---|
| Copper | 700 | μg | 900 | μg | 10000 | μg |

| | | | | | |
|---|---|---|---|---|---|
| Fluoride | NE | 4 | mg | 10 | mg |

| | | | | | | |
|---|---|---|---|---|---|---|
| Iodine | 95 | μg | 150 | μg | 1100 | μg |
| Iron | 6 | mg | 8 | mg | 45 | mg |
| Magnesium | 330 | mg | 400 | mg | 350 | mg |

| | | | | | |
|---|---|---|---|---|---|
| Manganese | NE | 2.3 | mg | 11 | mg |

| | | | | | | |
|---|---|---|---|---|---|---|
| Molybdenum | 34 | μg | 45 | μg | 2000 | μg |
| Phosphorus | 580 | mg | 700 | mg | 4000 | mg |

| | | | | |
|---|---|---|---|---|
| Potassium | NE | 4700 | mg | ND |

| | | | | | | |
|---|---|---|---|---|---|---|
| Selenium | 45 | μg | 55 | μg | 400 | μg |

| | | | | | |
|---|---|---|---|---|---|
| Sodium | NE | 1500 | mg | 2300 | mg |

| Vitamin A | 625 | μg | 900 | μg | 3000 | μg |
|---|---|---|---|---|---|---|
| Vitamin B1 | 1.0 | mg | 1.2 | mg | ND | |
| Vitamin B12 | 2.0 | μg | 2.4 | μg | ND | |
| Vitamin B2 | 1.1 | mg | 1.3 | mg | ND | |
| Vitamin B3 | 12 | mg | 16 | mg | 35 | mg |

| | | | | |
|---|---|---|---|---|
| Vitamin B5 | NE | 5 | mg | ND |

| | | | | | | |
|---|---|---|---|---|---|---|
| Vitamin B6 | 1.1 | mg | 1.3 | mg | 100 | mg |

| | | | | |
|---|---|---|---|---|
| Vitamin B7 | NE | 30 | μg | ND |

| | | | | | | |
|---|---|---|---|---|---|---|
| Vitamin B9 | 320 | μg | 400 | μg | 1000 | μg |
| Vitamin C | 75 | mg | 90 | mg | 2000 | mg |
| Vitamin D | 10 | μg | 15 | μg | 100 | μg |
| Vitamin E | 12 | mg | 15 | mg | 1000 | mg |

| | | | | |
|---|---|---|---|---|
| Vitamin K | NE | 120 | μg | ND |

| | | | | | | |
|---|---|---|---|---|---|---|
| Zinc | 9.4 | mg | 11 | mg | 40 | mg |

Preferably, the amount of a micro-nutrient, vitamin or dietary element in component C is at least about 10% of the RDA of that nutrient, and more preferably at least about 20% of the RDA. Also preferred are amounts representing from about 30% to about 100% of the RDA. Further preferred is a maximum amount corresponding to the UL for the respective nutrient.

### Component D

Component D comprises at least one amino acid, optionally in the form of a powder, a powder blend and/or a granulate. The definitions of amino acid(s) optionally comprised inside the ingestible particles as provided above equally apply to component D. The selection of the number, type and/or combination of the one or more amino acids in component D may be identical to that of the amino acid(s) optionally employed inside the ingestible particles. However, this is not a requirement; i.e. the ingestible particles may also contain different amino acid(s) than component D.

### Component E

Component E comprises one or more substance(s) for improved flavour, including but not limited to sweetening agents (such as sugars, sugar alcohols, stevia/steviosides etc.), bitterness reducing agents or flavouring agents such as natural, semisynthetic or synthetic aroma; plant extracts or powdered plant parts.

Flavouring agents for the purpose of the invention include, but are not limited to synthetic flavour oils and flavouring aromatics and/or natural oils, extracts from plants, leaves, flowers, and fruits, and mixtures of two or more thereof. These may include cinnamon oil, oil of wintergreen, peppermint oils, clove oil, bay oil, anise oil, eucalyptus, thyme oil, cedar leaf oil, oil of nutmeg, oil of sage, oils of citrus fruits (for example lemon and orange), oil of bitter almonds and cassia oil, vanilla, chocolate, mocha, coffee, ice cream, citrus (including lemon, orange, grape, lime, and grapefruit), apple, pear, peach, mango, strawberry, raspberry, cherry, plum, pineapple, and apricot. The amount of the at least one flavouring agents may depend on a number of factors, including the organoleptic effect desired.

Other Components

The pharmaceutical combination product may further comprise one or more additional components that may further contribute to its dietary effectiveness or health benefits; for example, non-fibrous prebiotics or omega fatty acid compounds. Further suitable additional components are γ-polyglutamic acid (γ-PGA), seaweed extract, isoflavones, green coffee extract, melon extract, carotenoids, docosahexaenoic acid, fish and krill oil, eicosapentaenoic acid, CoQ10, resveratrol, vegetable and fruit oils, caffeine, ephedra, capsicum, ginger, pyruvate, EGCS, taurine, polyphenols, herbal extracts; e. g. chamomile, lemon balm, passion flower, hops, valerian, theanine, lutein esters, lycopene, glucose, palatinose, taurine, ribose, guarana, glucuronolactone, citicoline, yeast beta-glucan, barley beta-glucan, oat beta-glucan, probiotics, plant sterols, tomato extract, chondroitin sulfate, collagen, biotin, electrolytes, and conjugated linoleic acid. Some of these components, such as fruit oils, may also be employed for their taste.

Other optional components or constituents may be present in the pharmaceutical combination product as well as the constituents thereof, such as a colouring agent, a stabilising agent, a wetting agent, a bulking agent, a suspending agent, a pH-modifying agent, and/or a flow-regulating agent.

Suitable colouring agents for the purpose of the invention include, but are not limited to, titanium dioxide and dyes suitable for food such as those known as FD&C dyes and natural colouring agents such as grape skin extract, beet red powder, beta-carotene, annatto, carmine, turmeric, chlorophyll, and pepper.

As mentioned, the pharmaceutical combination product always comprises the ingestible particles as defined above, the lipase inhibitor and optionally one or more of components A, B, C, D or E. The decision on how much of any one of the components A to E is to be added to the combination product is made independently for each component. One of the specific benefits of the combination product is that is can easily be adapted to the needs of an individual user or patient. An individual in need of e.g. preventing, controlling or reducing obesity or overweight will always benefit from the satiety-inducing effect of the ingestible particles, but at the same time may have different requirements with respect to the other components. For example, a person who wishes to replace a major meal with a single dose of the combination product on a regular basis, e.g. once a day for a certain period of time, may be interested in ensuring that such substitution will not lead to a lack of essential nutrient intake, such as the intake of protein, vitamins and dietary elements. If the replaced major meal is a protein-rich meal, the composition administered to replace it may also be enriched with protein, i.e. comprise component A, in particular if the other meals that are not replaced contain a low amount of protein. On the other hand, if the replaced meal is a light meal, a carbohydrate-rich meal or a snack, and the individual's regular intake of protein is not substantially affected by the meal replacement plan, then it may be more useful to incorporate component C in the composition. If the individual's change in diet tends to result in constipation, or if the health status of the individual indicates a need for—or potential benefit of—additional fibre intake, the composition may be designed to include component B.

Dietary and Therapeutic Uses

As mentioned, the ingestible particles and the combination products of the invention may be used for the suppression of appetite, in particular in human subjects, and for the induction of satiety in combination with the weight-loss-inducing effect of orlistat. Without wishing to be bound by theory, it is currently believed by the inventors that the appetite suppressing effect is based on the fatty acid compound comprised in the first lipid material of the ingestible particles, which upon ingestion interacts with physiological targets located in the mucosa of the gastrointestinal tract, such as in the stomach and/or duodenum, thereby activating one or more signalling cascades which eventually produce a perception of satiety or a reduction of appetite or hunger. Possibly, one of the targets at which the fatty acid acts are the ghrelin cells (or ghrelin receptors), large numbers of which are located in the stomach and the duodenum. The water-swellable or water-soluble polymeric material was found by the inventors to enhance the effect of at least the fatty acid (and optionally that of further components such as the lipase inhibitor, an amino acid, a vitamin, a micro-nutrient), which is possibly due to the swelling and/or mucoadhesive properties effecting a prolonged attachment of the particles (or components thereof) to the gastric or duodenal mucosa, allowing for an increased interaction of at least the fatty acid with the target structure. Of course, other properties of the particles may also effect or contribute to a prolonged gastric residence time, such as the selected particle size or the low density resulting from the high lipid content. In any case, the inventors found that the oral administration of the particles to volunteers induced satiety with the consequence that the subjects experienced suppressed appetite and showed a reduced food intake during the meal following the administration of a composition comprising the particles described herein. This effect was consistent with animal data showing the composition leads to a weight loss of the test animals.

For the embodiments where the ingestible particles further comprise one or more amino acid(s), the satiety inducing effect may be improved by said amino acids. The amino acids also benefit from the enhancing effects of the water-swellable or water-soluble polymeric material; i.e. prolonged attachment of the particles and increased interaction with the target structures.

The particles and/or compositions of the invention may therefore be used clinically, in combination with an oral lipase inhibitor such as orlistat, for the prevention or treatment of obesity and overweight, as well as the prevention or treatment of diseases or conditions associated with obesity (such as diabetes type 2) and/or with the use of lipase inhibitors (such as gastro-intestinal problems). Moreover, the use in appetite suppression and induction of satiety is provided. The use may be associated with a dietary schedule according to which a single dose of the composition is administered to a human subject at least once a day over a period of at least one week, and wherein optionally the human subject may be instructed to substitute a meal, partially or entirely, with said administration. As used herein, obesity is a medical condition in which excess body fat has accumulated to the extent that it may have an adverse effect on health. Overweight is understood as a borderline condition characterised by a body mass index (BMI) between 25 and below 30. Starting from a BMI of 30, the condition is classified as obesity.

Gastro-intestinal problems associated with the use of lipase inhibitors include steatorrhea (oily, loose stools with excessive flatus due to unabsorbed fats reaching the large intestine), faecal incontinence and frequent or urgent bowel movements.

In one embodiment, the particles and/or compositions are administered to normal weight or overweight subjects gaining weight over time or otherwise being at risk of developing obesity. In this case, the therapeutical objective is to stop or limit the weight gain and prevent the development of obesity. Another purpose may be to reduce the risk that the subject develops a disease or condition associated with or caused by obesity.

In a further embodiment, the particles and/or compositions are administered to obese patients in order to treat or reduce the severity of obesity. Again, the therapeutic use may also be directed to the reduction of the risk of developing a disease or condition associated with or caused by obesity.

A large number of diseases and conditions are nowadays considered to be associated with or caused by obesity, even though the mechanism by which they are linked to obesity may not always be fully understood. In particular, these diseases and conditions include—without limitation—diabetes mellitus type 2, arterial hypertension, metabolic syndrome, insulin resistance, hypercholesterolaemia, hypertriglyceridaemia, osteoarthritis, obstructive sleep apnoea, ischaemic heart disease, myocardial infarction, congestive heart failure, stroke, gout, and low back pain. The prevention or reduction of risk for developing any of these conditions is within the scope of the therapeutic use according to the invention.

Moreover, the therapeutic use preferably involves the at least once daily oral administration of the particles and/or compositions combined with an oral lipase inhibitor such as orlistat of the invention over a period of at least one week. In this context, the expression “therapeutic use” is understood to also cover the preventive or prophylactic use. In a further preferred embodiment, the particles and/or compositions combined with an oral lipase inhibitor such as orlistat are administered to a human subject over a period of at least about 2 weeks, or at least about 4 weeks, or at least about 6 weeks, or at least about 2 months, respectively. Also preferred is an administration regimen providing for once or twice or thrice daily administration.

The time of administration should be selected to maximise the lipase-inhibiting, steatorrhea-minimizing and satiety-inducing effects on the amount of food which is subsequently taken up by the subject that is treated. For example, it is useful to administer a dose of the pharmaceutical combination product according to the invention before a major meal, such as before a lunchtime meal and/or before the evening dinner such as to reduce the amount of food eaten during either of these meals. It may also be useful to administer a dose of the pharmaceutical combination product according to the invention three times a day, such as before breakfast, before a lunchtime meal and before an evening dinner. With respect to the precise timing, it is preferred that the dose is administered within about 5 to 120 minutes prior to the respective meal, in particular about 10 to about 120 minutes prior to the meal, or about 15 to about 90 minutes prior to the meal, such as about 30 or about 60 minutes prior to the meal.

In one of the particularly preferred embodiments, a dose comprising at least about 5 g of the first lipid material is administered to a human subject at least once daily between about 15 and about 90 minutes prior to a meal over a period of at least 4 weeks for the prevention or treatment of obesity or an associated disease. This dose further comprises 200 mg of a lipase inhibitor such as orlistat or less; e.g. 120 mg, 80 mg, 60 mg, 40 mg or 20 mg orlistat.

In respect of the adaptability of the combination product by additional components A to D, the invention further provides a method of preventing, controlling or treating obesity or overweight in an individual, such method comprising the steps of

(a) providing the ingestible particles and at least components A, B, C, and D separately, e.g. in dry, flowable form,

(b) determining the dietary needs of the individual, taking into account the changes in the daily intake of nutrients foreseen by meal replacement,

(c) combining a single dose of the ingestible particles, or of a solid composition comprising them, with a single dose of at least one of the components A, B, C and/or D according to the dietary needs as determined in step (b) into a single dose of the composition, and

(d) administering the single dose of the composition to the individual.

In one embodiment, step (d) is performed on a continuous basis, preferably at least once a day over a period of at least one week. Preferably, the administration is conducted after reconstitution of the combination product into a drinkable suspension. In one embodiment, the individual replaces a meal with an administration of a single dose of the combination product.

Of course, more than one meal per day may be replaced. For example, an individual may decide, or be instructed, to replace, on a daily basis, a sweet, carbohydrate-rich, low-protein breakfast as well as dinner, taking a single dose of the combination product in the morning and another single dose in the evening. In such a diet plan, it may be appropriate to use different compositions, e.g. a low-protein but fibre-rich composition with vitamins and dietary elements (i.e. a composition comprising components B and C, with little or no component A and D) to substitute breakfast (partially or entirely), and a composition with substantial protein content (i.e. component A along with the ingestible particles) to substitute the dinner (partially or entirely), both compositions being satiety-inducing due to the presence of the ingestible particles.

It is further contemplated that the particles and/or combination products of the invention are used in combination with the use of a device for the collection, storage and/or display of information relating to a subject's adherence to the therapy and/or the effectiveness of the therapy. As used herein, information relating to a subject's adherence to the therapy may include, for example, information on whether a dose was administered within a certain period of time (e.g. during a calendar day), or the time at which each dose was administered. The device is preferably a programmed electronic device, such as a computer, in particular a microcomputer, and most preferably a portable microcomputer such as a mobile phone (“smartphone”), or a wearable device such as a smart watch, an electronic wristband, or the like. The information may be received by the device automatically from a sensor, or it may be entered manually by a user, such as the subject or patient, the physician, nurse, or by a caregiver, and stored for subsequent analysis or display. For example, the patient may periodically monitor his or her actual compliance or adherence to the therapy.

The device may be programmed to provide the user with a feedback signal or reminder in case of non-compliance or lack of adequate adherence to the therapy. The feedback signal may be optical, haptic (e.g. vibration), or acoustic.

Information relating to the effectiveness of the therapy may include, for example, the weight of the subject, the degree of hunger or appetite, the number of meals and snacks, or the type or amount of food eaten during any particular period of time (e.g. a calendar day), or even physiological data such as the blood glucose concentration or blood pressure. Depending on its type, the information relating to the effectiveness of the therapy may be automatically received by the device or entered manually by the user. Information with respect to the feeling of satiety or hunger may be usefully entered by the user or patient in a manual mode, whereas physiological parameters such as blood glucose or blood pressure may be received from the respective measuring devices used for their determination. In the latter case, the transfer of the data encoding the information generated by the measuring device to the device for the storage and/or display of the information is preferably wireless.

In more detail, information collection may be user-initiated or the device may be programmed with an application (i.e. software) which creates an alert calling for the user to input her or his satiety-state information. Preferably, information collection proceeds in regular time intervals such as 15 or 30 min intervals. In one embodiment, information collection is performed throughout a period of 12, 16 or 18 hours per day. In another embodiment, information collection is performed in multiple periods of for instance 1 to 3 hours over the day, for instance three times for 3 hours each. Preferably such time periods cover meal times such as breakfast, lunch and dinner. Preferably, users—for a given period of information collection—may not refer to previous satiety ratings when providing the real-time information.

Information collection may proceed in the following fashion. After the user has opened the software application, a satiety state screen is displayed on the colour touch screen using visual analogue scales for the assessment of satiety. Such scales and scores have previously been described in detail [Flint A, Raben A, Blundell J E, Astrup A. Reproducibility, power and validity of visual analogue scales in assessment of appetite sensations in single test meal studies. Int J Obes Relat Metab Disord 2000; 24:38-48). In brief, the visual analogue scale (VAS) consists of a horizontal, unstructured, 10 cm line with words anchored at each end, describing the extremes (‘not at all’ or ‘extremely’) of the unipolar question, ‘How satiated are you right now?’ To ensure reliable and valid results, participants rate their feeling of satiation as precisely as possible, and they cannot refer to their previous ratings when marking the VAS.

The satiety state screen may display a query 1 “how hungry do you feel?” combined with an unstructured sliding scale labelled “I am not hungry at all” on one end to “very hungry” on the other hand. The application will wait for the user to touch the sliding scale at one position. Upon touching the scale, a slider may appear, and the user may adjust its position. The application will determine the position of the slider after the user removed its touching finger from the slider symbol, retrieve the positional value and use it for further processing.

Further potentially useful embodiments are easily derivable on the basis of the guidance provided herein-above and the following examples.

### Examples

### Example 1: Preparation of Particles by Spray Congealing

Particles with a water-swellable or water-soluble polymeric material embedded within a lipid material may be prepared by spray congealing as follows. 250 g of capric acid are melted. 100.0 g of carbomer homopolymer type A NF and 50.0 g of sodium caprate are added to the melt and mixed such as to form a viscous suspension. Under continuous heating, the suspension is fed to the heated rotary nozzle of a spray congealing tower. Cold air is continuously introduced into the tower to allow solidification of the resulting droplets. The solid particles are then passed through appropriate sieves to allow removal of oversize and undersize particles, and to obtain particles according to the invention. Optionally, the product may be further processed, e.g. by coating the particles.

The product may further be provided as a combination product for oral administration together with a lipase inhibitor such as orlistat; e.g. by filling the spray congealed particles and lipase inhibitor into stick packs or sachets.

The product may yet further be provided as a combination product for oral administration together with a lipase inhibitor such as orlistat and other dry powderous components weighed out and combined in screw-top bottles (approx. 150-250 mL) and mixed by shaking the closed bottle. For instance:

Combination 1: 12.5 g lipid granules and 30 g soy protein concentrate (Soy Protein Isolate, myprotein, UK)

Combination 2: 12.5 g lipid granules and 30 g pea protein concentrate (Pea Protein Isolate, myprotein, UK)

Combination 3: 12.5 g lipid granules and 25 g whey protein concentrate (Impact Whey Protein, myprotein, UK)

Combination 4: 12.5 g lipid granules and 25 g whey protein preparation (Protein Smoothie, myprotein, UK) containing whey protein concentrate (74%), natural banana flavouring, natural strawberry flavouring, banana powder, strawberry powder, colour (curcumin, beetroot red), sweetener sucralose, soy lecithin.

Combination 5: 12.5 g lipid granules and 30 g brown rice protein concentrate (myprotein, UK)

Combination 6: 12.5 g lipid granules and 56 g soy protein preparation (Diät Vitalkost, DM, Germany) comprising soy protein isolate (40.5%), honey (20%), skimmed milk powder (12%), yoghurt powder (6%), maltodextrin, soy oil, inulin, milk protein, di-potassiumphosphate, tri-calciumphosphate, silicon dioxide, magnesium hydroxite, soy lecithin, L-ascorbic acid, iron-(III)-diphosphate, steviol glycoside, niacin, DL-alpha-tocopherol, zinc oxide, manganese-(II)-sulphate, copper carbonate, calcium-D-panthotenate, colouring beta-carotene, pyrodixine hydrochloride, thiaminmononitrate, riboflavin, retinylacetate, pteroylmonoglutamic acid, potassium iodide, sodium selenite, D-biotin, cholecalciferol, cyanocobalamin.

Combination 7: 12.5 g lipid granules and 50 g soy protein preparation (Almased, Germany) comprising soy protein (50%), bee honey (25%), skimmed milk-yoghurt powder (23%), potassium chloride, magnesium citrate, silicic acid, calcium citrate, vitamin C, Niacin, colouring riboflavin, vitamin E, zinc oxide, iron fumarate, manganese sulphate, calcium-D-panthotenate, vitamin B2, vitamin B6, vitamin B1, 274 μg vitamin A, folic acid, potassium iodide, sodium selenite, biotin, vitamin D3, vitamin B12.

Combination 8: 12.5 g lipid granules and 30 g whey protein preparation (Slim System, WPT, Germany) comprising protein-enriched whey powder, soy protein isolate, milk protein, wheat protein, flavour, carboxymethyl cellulose; L-carnitine, maltodextrin, sodium cyclamate, sodium saccharin, magnesium hydroxide, palm oil, ferric pyrophosphate, vitamin C, DL-a-Tocopherylacetate, nicotinamide, silicon dioxide, zinc oxide, riboflavin, calcium-D-pantothenate, manganese sulphate, cupric carbonate, cholecalciferol, pyridoxine hydrochloride, thiamin mononitrate, retinyl acetate, beta-carotene, folic acid, sodium selenite, sodium iodide, D-biotin, cyanocobalamin.

### Example 2: Preparation of Particles by Melt Extrusion

Lipid Granulates 2.1:

14 kg of a premix were prepared in seven batches of 2 kg each. For each batch, 0.9 kg palm stearin (Prifex® 300, Brenntag B.V., Belgium) and 0.1 kg linseed oil (manako BIO Leinol human, Makana, Germany) were brought to a melt in a cooking pot over an induction plate. When the melt had a temperature of 60° C., 0.3 kg sodium alginate (Alginex®, Kimica, Japan), 0.1 kg oat fibre preparation (PromOat®, Harke Pharma, Germany) and 0.1 kg pectin (Aglupectin® HS-RVP, NRC, Germany) were incorporated by means of a cooking spoon. The mixture was transferred in aliquots into zip-loc plastic bags and cooled to room temperature to form solid plates. Lipid-polymer plates were further cooled in a freezer set at −18° C. and then shredded to particles of approx. 5 mm and smaller by means of a blender (Vitamix® Professional 750, Vita-Mix Corp., USA). The obtained premix was fed via a volumetric dosing system (Dosimex DO-50, Gabler GmbH & Co KG, Germany) into a powder inlet of a twin screw extruder (Extruder DE-40/10, Gabler GmbH & Co KG, Germany) operating at 10 rpm and extruded at a temperature range of approx. 30° C. to strands of 1.0 mm diameter. Extruded strands were cut to granules of 0.8 mm to2.5 mm length by means of rotating blades running at 100 rpm. The extrudate was transferred into plastic bags in aliquots and stored at −18° C. Subsequently, granules were subjected to classification using wire mesh sieves (Atechnik GmbH, Germany) of 2 mm (mesh 10) and 1.0 mm (mesh 18). Material retained on the 2 mm sieve was subjected to comminution using a household blending device (MK55300, Siemens, Germany) and re-classified using the set of wire mesh sieves. Granules classified to a range of 1-2 mm were combined to give a yield of 9.0 kg and split into aliquots of 600 g.

Subsequently, batches (one aliquot per run, fifteen runs) were loaded into a fluid bed coating device (Ventilus V-2.5/1, Innojet, Germany, equipped with an IPC3 product reservoir) and fluidized at a bed temperature of 20° C. and an air flow of 65 m³/h. Per run, 120 g palm stearin (Prifex® 300, Brenntag N.V., Belgium) were molten in a beaker on a hot plate (at 100° C.) equipped with an overhead stirrer. The hot melt was quantitatively sprayed onto the granules using a peristaltic pump and a bottom spraying procedure at a spray rate of 6.5 g/min. Batches were combined, and a total of 10.67 kg of coated granules were obtained and stored in a plastic container.

Lipid Granulates 2.2:

14 kg of a premix were prepared in seven batches of 2 kg each. For each batch, 0.63 kg palm stearin (Prifex® 300, Brenntag B.V., Belgium) and 0.07 kg linseed oil (manako BIO Leinol human, Makana, Germany) were brought to a melt in a cooking pot over an induction plate. When the melt had a temperature of 60° C., 0.21 kg sodium alginate (Alginex®, Kimica, Japan), 0.07 kg oat fibre preparation (PromOat®, Harke Pharma, Germany), 0.07 kg pectin (Aglupectin® HS-RVP, NRC, Germany) and 0.35 kg resistant dextrin (Nutriose® FB06, Barentz, Germany) were incorporated by means of a cooking spoon. The mixture was transferred in aliquots into zip-loc plastic bags and cooled to room temperature to form solid plates. Lipid-polymer plates were further cooled in a freezer set at −18° C. and then shredded to particles of approx. 5 mm and smaller by means of a blender (Vitamix® Professional 750, Vita-Mix Corp., USA). The obtained premix was fed via a volumetric dosing system (Dosimex DO-50, Gabler GmbH & Co KG, Germany) into a powder inlet of a twin screw extruder (Extruder DE-40/10, Gabler GmbH & Co KG, Germany) operating at 10 rpm and extruded at a temperature range of approx. 30° C. to strands of 1.0 mm diameter. Extruded strands were cut to granules of 0.8 mm to 2.5 mm length by means of rotating blades running at 100 rpm. The extrudate was transferred into plastic bags in aliquots and stored at −18° C. Subsequently, granules were subjected to classification using wire mesh sieves (Atechnik GmbH, Germany) of 2 mm (mesh 10) and 1.0 mm (mesh 18). Material retained on the 2 mm sieve was subjected to comminution using a household blending device (MK55300, Siemens, Germany) and re-classified using the set of wire mesh sieves. Granules classified to a range of 1-2 mm were combined to give a yield of 9.0 kg and split into aliquots of 600 g.

Subsequently, batches (one aliquot per run, fifteen runs) were loaded into a fluid bed coating device (Ventilus V-2.5/1, Innojet, Germany, equipped with an IPC3 product reservoir) and fluidized at a bed temperature of 20° C. and an air flow of 65 m³/h. Per run, 120 g palm stearin (Prifex® 300, Brenntag N.V., Belgium) were molten in a beaker on a hot plate (at 100° C.) equipped with an overhead stirrer. The hot melt was quantitatively sprayed onto the granules using a peristaltic pump and a bottom spraying procedure at a spray rate of 6.5 g/min. Batches were combined, and a total of 10.67 kg of coated granules were obtained and stored in a plastic container.

Lipid Granulates 2.3:

12.5 kg glycerol monolaurate (GML 90 food, Mosselman, Belgium), 8.33 kg glycerol monooleate (Imwitor® 990, NRC, Germany) and 12.5 kg triglyceride (Witepsol® E85, NRC, Germany) were filled into a ploughshare batch mixer (FM130, Lodige Maschinenbau GmbH, Germany). The chamber was heated using an external temperature control system (Compact TKN-90-18-35, Single Temperiertechnik GmbH, Germany) with the mixing tool running at 40 rpm. After the lipid components were brought to a homogeneous melt at 60° C., 15.83 kg HPMC (AnyAddy® CN10T, Harke Pharma, Germany) and 0.83 kg xanthan (NRC, Germany) were added and blended at 40 rpm until homogeneity. Then, the heating system was turned off and 15 kg of dry ice were rapidly introduced into the mixing chamber with the mixer running at 50 rpm. Subsequently, the milling head (speed 2) was activated and granulate premix was obtained, released through the outlet and collected in bags.

The obtained premix was fed via a volumetric dosing system (Dosimex DO-50, Gabler GmbH & Co KG, Germany) into a powder inlet of a twin screw extruder (Extruder DE-40/10, Gabler GmbH & Co KG, Germany) operating at 15 rpm and extruded at a temperature range of approx. 18° C. to strands of 1.0 mm diameter. Extruded strands were cut to granules of 0.8 mm to 2.5 mm length by means of rotating blades running at 350 rpm. Subsequently, the extrudate was classified on a sieving machine (Siftomat 1, Fuchs Maschinen AG, Switzerland) to collect granules of 1-2 mm.

45 kg of the resulting extrudate material was loaded into a fluid bed coating device (Ventilus V-100, Innojet, Germany) and fluidized at a bed temperature of 15° C. and an air flow of 1000 m³/h. 9 kg hard fat (Dynasan® 116, Cremer Oleo GmbH & Co KG, Germany) were molten in an external heating system and the hot melt was quantitatively sprayed onto the granulate using hot melt bottom spraying system at a pressure of 1.2 bar and a spray rate of 300 g/min. Coated granules were obtained and stored in a plastic container.

Lipid Granulates 2.4:

22.5 kg triglyceride (Witepsol® W25, NRC, Germany) were filled into a ploughshare batch mixer, (FM130, Gebrüder Lödige Maschinenbau GmbH, Germany). The chamber was heated using an external temperature control system (Compact TKN-90-18-35, Single Temperiertechnik GmbH, Germany) with the mixing tool running at 40 rpm. After the lipid components were brought to a homogeneous melt at 38° C., 27.0 kg sodium alginate (Satialgine®, Overlack, Germany) were added and blended at 40 rpm until homogeneity. Then, the heating system was turned off and 10 kg of dry ice were rapidly introduced into the mixing chamber with the mixer running at 50 rpm. Subsequently, the milling head (speed 2) was activated and granulate premix was obtained, released through the outlet and collected in bags.

The obtained premix was fed via a volumetric dosing system (Dosimex DO-50, Gabler GmbH & Co KG, Germany) into a powder inlet of a twin screw extruder (Extruder DE-40/10, Gabler GmbH & Co KG, Germany) operating at 15 rpm and extruded at a temperature range of approx. 27° C. to strands of 1.0 mm diameter. Extruded strands were cut to granules of 0.8 mm to 2.5 mm length by means of rotating blades running at 350 rpm. Subsequently, the extrudate was classified on a sieving machine (Siftomat 1, Fuchs Maschinen AG, Switzerland) to collect granules of 1-2 mm.

45 kg of the resulting extrudate material was loaded into a fluid bed coating device (Ventilus V-100, Innojet, Germany) and fluidized at a bed temperature of 15° C. and an air flow of 1000 m³/h. 9 kg hard fat (Dynasan® 116, Cremer Oleo GmbH & Co KG, Germany) were molten in an external heating system and the hot melt was quantitatively sprayed onto the granulate using hot melt bottom spraying system at a pressure of 1.2 bar and a spray rate of 300 g/min. Coated granules were obtained and stored in a plastic container.

Orlistat Combination Product 2.5:

In a 150 mL screw-top bottle, 60 mg orlistat micro-pellets (contents of one capsule of Orlistat® Hexal, Hexal, Germany) were combined with:
15.4 g ingestible particles of granulate 2.3,
4 g resistant dextrin (Nutriose® FB06, Barentz, Germany),
1.25 g psyllium husk powder (Carepsyllium 99/100, Caremoli, Italy) and
7 g whey protein preparation (Slim System, WPT, Germany; comprising protein-enriched whey powder, soy protein isolate, milk protein, wheat protein, flavour, carboxymethyl cellulose, L-carnitine, maltodextrin, sodium cyclamate, sodium saccharin, magnesium hydroxide, palm oil, ferric pyrophosphate, vitamin C, DL-a-Tocopherylacetate, nicotinamide, silicon dioxide, zinc oxide, riboflavin, calcium-D-pantothenate, manganese sulphate, cupric carbonate, cholecalciferol, pyridoxine hydrochloride, thiamin mononitrate, retinyl acetate, beta-carotene, folic acid, sodium selenite, sodium iodide, D-biotin, cyanocobalamin).

### Example 3: Comparison of High Fat Diet Effects Under Orlistat Versus Orlistat and Polyacrylic Acid (PAA); Orlistat and Resistant Dextrin; or Orlistat and HPMC/Xanthan

General Procedures:

Animals (male rats) were kept in cages on standard animal bedding (two animals per cage or individual housing) and were provided with ad libitum access to food and water. Animal food was provided as pellets in a pellet rack or as a cream or as granulate powder each filled in a container attached to the inside of the cage.

Body weight was recorded at beginning and end of experiments. Food consumption was documented daily except for weekends. Experiments were performed according to German laws of animal protection.

Rodent chow was purchased from ssniff Spezialdiaten GmbH, Germany; poly(acrylic acid) (PAA, Carbopol® 971 P NF) was obtained from the Lubrizol Corporation, USA; and HPMC (AnyAddy®) was obtained from Harke Pharma, Germany. Orlistat (Hexal, Germany) was purchased in a local pharmacy. Hard fat/tripalmitin (Dynasan® 116), triglyceride (Witepsol® E85), glycerol monooleate (Imwitor®990), and Xanthan were obtained from NRC/Jungbunzlauer, Germany. Glycerol monolaurate (GML 90 food) was obtained from Mosselman, Belgium. Resistant dextrin (Nutriose® FB06) was obtained from Barentz, Germany or Barentz, Netherlands.

Preparation of Lipid Containing Granulates:

Lipid Granulates A or B:

Lipid granules A or B were produced by bringing a 1:1 (w/w) mixture of glycerol monolaurate (GML 90 food) and tripalmitin (Dynasan® 116) to a homogenous melt in a cooking pot by means of a heating plate. Polyacrylic acid PAA (lipid granulate A; Carbopol® 971 P NF) or resistant dextrin (lipid granulate B; Nutriose® FB06),—each at 50 wt-% of the lipid mixture, respectively—were added to the melt and incorporated by mechanical mixing. The compositions were poured into zip-loc-bags and cooled to −18° C. in a freezer. The frozen material was first crushed by means of a hammer and then shredded to a granulate in a kitchen blender (Vitamix® Professional 750, Vita-Mix Corp., USA), and classified through a set of wire mesh sieves (VWR International, Germany) to a granulate size of below 2.0 mm and above 1.3 mm.

Lipid Granulate C:

12.5 kg glycerol monolaurate (GML 90 food), 8.33 kg glycerol monooleate (Imwitor® 990) and 12.5 kg triglyceride (Witepsol® E85) were filled into a ploughshare batch mixer (FM130, Lödige Maschinenbau GmbH, Germany). The chamber was heated using an external temperature control system (Compact TKN-90-18-35, Single Temperiertechnik GmbH, Germany) with the mixing tool running at 40 rpm. After the lipid components were brought to a homogeneous melt at 60° C., 15.83 kg HPMC (AnyAddy® CN10T) and 0.83 kg Xanthan were added and blended at 40 rpm until homogeneity. Then, the heating system was turned off and 15 kg of dry ice were rapidly introduced into the mixing chamber with the mixer running at 50 rpm. Subsequently, the milling head was activated and granulate premix was generated, released through the outlet and collected in bags.

The obtained premix was fed via a volumetric dosing system (Dosimex DO-50, Gabler GmbH & Co KG, Germany) into a powder inlet of a twin screw extruder (Extruder DE-40/10, Gabler GmbH & Co KG, Germany) operating at 15 rpm and extruded at a temperature range of approx. 18° C. to strands of 1.0 mm diameter. Extruded strands were cut to granules of 0.8 mm to 2.5 mm length by means of rotating blades running at 350 rpm. Subsequently, the extrudate was classified on a sieving machine (Siftomat 1, Fuchs Maschinen AG, Switzerland) to collect granules of 1-2 mm.

45 kg granulate were then loaded into a fluid bed coating device (Ventilus V-100, Innojet, Germany) and fluidized at a bed temperature of 15° C. and an air flow of 1000 m³/h.
9 kg hard fat (Dynasan® 116, NRC, Germany) were molten in an external heating system and the hot melt was quantitatively sprayed onto the granulate using hot melt bottom spraying system at a pressure of 1.2 bar and a spray rate of 300 g/min. Coated granules were collected and stored in a plastic container.

Feeding Experiments:

Ex. 3.1.: Cream Chow with 50% Fat (Orlistat Free Control for High Fat Diet)

Four male wistar rats having a mean body weight of 329 g were fed an experimental high-fat chow (EF R/M comprising 50% fat) provided as cream. At the end of the experiment, body weight change was evaluated (±SD). Animals gained 5.0±1.9% body weight; mean daily food intake was 15.0 g. Faeces were well formed and mostly hard and dry when collected.

Ex. 3.2.: Cream Chow with 50% Fat and Orlistat (Orlistat Control)

Four male wistar rats having a mean body weight of 357 g were fed an experimental high-fat cream chow (EF R/M comprising 50% fat) with added orlistat (0.6 mg/g chow) for seven days. At the end of the experiment, body weight change was evaluated (±SD). Animals lost 2.5±1.4% body weight; mean daily food intake was 23.6 g. All animals had developed severe steatorrhea with amorphous and semi-liquid faeces.

Ex. 3.3.: Cream Chow with 50% Fat and Orlistat and Polyacrylic Acid Powder (PAA)

Four male wistar rats having a mean body weight of 339 g were fed an experimental high-fat cream chow (EF R/M comprising 50% fat) with added orlistat (0.6 mg/g chow) and 6 wt.-% PAA (Carbopol) for thirteen days. At the end of the experiment, body weight change was evaluated (±SD). Animals lost 7.6±3.8% body weight; mean daily food intake was 26.3 g. All animals exhibited voluminous well-formed elastic faeces. No signs of steatorrhea were observed.

Ex. 3.4.: Cream Chow with 53% Fat and Orlistat and Lipid Granulate A (PAA)

Twelve male wistar rats having a mean body weight of 306 g were fed an experimental high-fat cream chow (EF R/M comprising 50% fat) with added orlistat (0.6 mg/g chow) and 18 wt.-% lipid granulate A for seven days. The total amount of fat in the cream chow was 53 wt.-% and total amount of PAA was 6 wt.-%. At the end of the experiment, body weight change was evaluated (±SD). Animals lost 3.9±5.0% body weight: mean daily food intake was 24.0 g. All animals exhibited voluminous well-formed elastic faeces. No signs of steatorrhea were observed.

Ex. 3.5.: Cream Chow with 53% Fat and Orlistat and Lipid Granulate B (Resistant Dextrin)

Twelve male wistar rats having a mean body weight of 312 g were fed an experimental high-fat cream chow (EF R/M comprising 50% fat) with added orlistat (0.6 mg/g chow) and 18 wt.-% lipid granulate B for seven days. The total amount of fat in the cream chow was 53 wt.-% and total amount of resistant dextrin was 6 wt.-%. At the end of the experiment, body weight change was evaluated (±SD). Animals gained 0.1±2.8% body weight; mean daily food intake was 29.3 g. All animals exhibited voluminous well-formed elastic faeces. No signs of steatorrhea were observed.

Ex. 3.6.: Cream Chow with 54% Fat and Orlistat and Lipid Granulate C (HPMC/Xanthan)

Twelve male wistar rats having a mean body weight of 364 g were fed an experimental high-fat cream chow (EF R/M comprising 50% fat) with added orlistat (0.6 mg/g chow) and 22 wt.-% lipid granulate C for seven days. The total amount of fat in the cream chow was 54 wt.-% and total amount of HPMC and xanthan was 6 wt.-%. At the end of the experiment, body weight change was evaluated (±SD). Animals lost 1.4±2.1% body weight; mean daily food intake was 27.7 g. All animals exhibited voluminous well-formed elastic faeces. No signs of steatorrhea were observed.

1. A pharmaceutical combination product for oral administration comprising:
(i) a lipase inhibitor; and
(ii) a plurality of ingestible particles having a sieve diameter in the range from 0.01 mm to 10 mm, or from 0.05 mm to 3 mm, said particles comprising at least
(a) a water-swellable or water-soluble polymeric material, and
(b) a first lipid material;
wherein
the first lipid material comprises a medium or long chain fatty acid compound, and
the water-swellable or water-soluble polymeric material is embedded within, and/or coated with, the lipid material.

2. The pharmaceutical combination product of claim 1, wherein
(a) the lipase inhibitor is contained in the ingestible particles; and/or
(b) the lipase inhibitor is provided separately from the ingestible particles.

3. The pharmaceutical combination product of claim 2, wherein the lipase inhibitor is provided separately from the ingestible particles and in the same pharmaceutical composition as the ingestible particles.

4. The pharmaceutical combination product of claim 3, wherein the lipase inhibitor and the plurality of ingestible particles are mixed and compressed into tablets or filled into capsules, sachets, stick packs, bottles or containers.

5. The pharmaceutical combination product of claim 2, wherein the lipase inhibitor is provided separately from the ingestible particles, and in a separate pharmaceutical composition, said separate pharmaceutical composition being provided together with the plurality of ingestible particles in the form of a kit.

6. The pharmaceutical combination product of claim 1, wherein the lipase inhibitor is orlistat.

7. The pharmaceutical combination product of claim 1, wherein the ingestible particles comprise an active core and a coating, wherein
the active core comprises the water-swellable or water-soluble polymeric material and the first lipid material,
the coating comprises a second lipid material and/or a hydrophilic material, wherein the coating may be substantially free of the water-swellable or water-soluble polymeric material, and
wherein the composition of the second lipid material may be the same as, or different from, the composition of the first lipid material.

8. The pharmaceutical combination product of claim 1, wherein the ingestible particles comprise
an inert core,
a first coating covering the inert core, and
a second coating covering the first coating,
wherein the first coating comprises the water-swellable or water-soluble polymeric material and the first lipid material, and
wherein the second coating comprises a second lipid material and/or a hydrophilic material,
wherein the second coating is substantially free of the water-swellable or water-soluble polymeric material, and
wherein the composition of the second lipid material may be the same as, or different from, the composition of the first lipid material.

9. The pharmaceutical combination product of claim 1, wherein the water-swellable or water-soluble polymeric material of the ingestible particles comprises at least one polymeric material selected from poly(carboxylate), chitosan, cellulose ethers, and xanthan gum; and
wherein the poly(carboxylate) is preferably selected from alginic acid, sodium alginate, pectin, poly(acrylic acid), poly(methacrylic acid), copolymers of acrylic and methacrylic acid, poly(hydroxyethyl methacrylic acid);
wherein the cellulose ether is preferably selected from hydroxyethyl cellulose, hydroxypropyl cellulose, hydroxypropyl methylcellulose, methylcellulose, and carboxymethylcellulose;
wherein the poly(carboxylate) and/or the carboxymethylcellulose is optionally at least partially neutralised; and
wherein the polymeric material is optionally at least partially crosslinked.

10. The pharmaceutical combination product of claim 9, wherein the first lipid material component comprises at least one medium or long chain fatty acid compound with a melting range below 37° C., or at least one medium or long chain fatty acid compound with a melting range above 37° C., or a mixture thereof; and/or
wherein further the content of di- and triglycerides within the first lipid material is 80% or less, preferably 50% or less.

11. The pharmaceutical combination product of claim 1,
wherein the ingestible particles are provided in the form of granules, pellets, or minitablets.

12. An ingestible particle having a sieve diameter in the range from 0.01 mm to 10 mm, or from 0.05 to 3 mm, said particle comprising
(a) a water-swellable or water-soluble polymeric material,
(b) a first lipid material; and
(c) a lipase inhibitor; and optionally
(d) an amino acid, a vitamin, a micro-nutrient, or any combination thereof; wherein
the first lipid material comprises a medium or long chain fatty acid compound, and
the water-swellable or water-soluble polymeric material is embedded within, and/or coated with, the lipid material.

13. The ingestible particle of claim 12, wherein the water-swellable or water-soluble polymeric material of the ingestible particles comprises polyacrylic acid.

14. The pharmaceutical combination product of claim 1 for use in the prevention and/or treatment of obesity or a disease or condition associated with obesity and/or the use of lipase inhibitors.

15. The pharmaceutical combination product of claim 1 for use in the prevention and/or treatment of lipase inhibitor induced gastro-intestinal problems.

16. The ingestible particle of claim 12 for use in the prevention and/or treatment of obesity or a disease or condition associated with obesity and/or the use of lipase inhibitors.

17. The ingestible particle of claim 12 for use in the prevention and/or treatment of lipase inhibitor induced gastro-intestinal problems.
